(19) United States
(12) Patent Application Publication
(10) Pub. No.: US 2024/0409498 A1
(43) Pub. Date: Dec. 12, 2024

(54) PROCESS OF MAKING 3-(4'-AMINOPHENYL)-2-METHOXYPROPIONIC ACID, AND ANALOGS AND INTERMEDIATES THEREOF

(71) Applicant: Nogra Pharma Limited, Dublin 2 (IE)

(72) Inventors: Salvatore Demartis, Milan (IT); Francesca Viti, Salorino (CH); Marie McNulty, Dublin (IE)

(21) Appl. No.: 18/409,063

(22) Filed: Jan. 10, 2024

Related U.S. Application Data
(63) Continuation of application No. 17/429,183, filed on Jul. 14, 2022, now Pat. No. 11,905,232.
(60) Provisional application No. 62/802,802, filed on Feb. 8, 2019.

(51) Int. Cl. C07C 231/10; C07C 201/12; C07C 205/56; C07C 227/04; C07C 231/02; C07C 231/18; C07C 233/54; C07C 303/28

(52) U.S. Cl. CPC C07C 231/10; C07C 201/12; C07C 205/56; C07C 227/04; C07C 231/02; C07C 231/18; C07C 233/54; C07C 303/28

(57) ABSTRACT

The disclosure provides a process for the preparation of 3-(4′-annitophenyl)-2-metlioxypropionic acid, and analogs and intermediates thereof, contemplated to be capable of modulating the activity of receptors, e.g., PPARs receptors.

### BACKGROUND

Peroxisome Proliferator Activated Receptors (PPARs) are members of the nuclear hormone receptor super family, which are ligand-activated transcription factors regulating gene expression. Certain PPARs play roles in the regulation of cell differentiation, development and metabolism of higher organisms.

Three types of PPAR have been identified: alpha, expressed in the liver, kidney, heart and other tissues and organs, beta/delta expressed, for example, in the brain, and gamma, expressed in three forms: gamma1, gamma2, and gamma3. PPARγ receptors have been associated with a number of disease states including fibrotic diseases, dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, TBDs (irritable bowel disease), ulcerative colitis and Crohn's disease.

Further, treatment of tumor cells with ligands of PPARγ receptors can induce a decrease in cellular proliferation, cell differentiation and apoptosis, and therefore may be useful in preventing carcinogenesis. Intestinal anti-inflammatory activity may be dependent on binding and subsequent activation of PPARγ receptors.

Accordingly, effective processes for making compounds capable of modulating the activity of PPARs receptors are needed to address the treatment of such diseases.

### SUMMARY

The disclosure provides, for example, a process for the preparation of compounds which may be modulators of PPARs receptors.

The present disclosure provides, in part, a process for the preparation of a compound of Formula (VII):

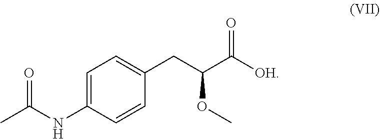 [SMILES: CO[C@@H](Cc1ccc(NC(C)=O)cc1)C(=O)O]

One embodiment provides a process for preparing of a compound of Formula (VI):

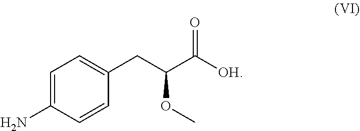

Also contemplated herein is a process for the preparation of analogs and intermediates thereof.

In one embodiment, at least some of the compounds identified as intermediates e.g., as part of a synthetic scheme disclosed herein are contemplated as compounds of the disclosure, e.g., a compound represented by Formula (I) or a pharmaceutically acceptable salt or stereoisomer thereof:

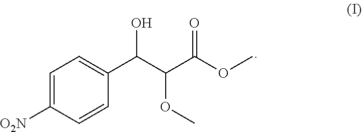 [SMILES: COC(=O)C(OC)C(O)c1ccc([N+](=O)[O-])cc1]

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### DEFINITIONS

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, and C₂-C₆alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, etc.

The term “alkoxy” as used herein refers to an alkyl group attached to an oxygen (—O—alkyl). Exemplary alkoxy groups include, but are not limited to, groups with an alkyl, alkenyl or alkynyl group of 1-12, 1-8, or 1-6 carbon atoms, referred to herein as C₁-C₁₂alkoxy, C₁-C₈alkoxy, and C₁-C₆alkoxy, respectively. Exemplary alkoxy groups include, but are not limited to methoxy, ethoxy, etc. Similarly, exemplary “alkenoxy” groups include, but are not limited to vinyloxy, allyloxy, butenoxy, etc.

The term “alkyl” as used herein refers to a saturated straight or branched hydrocarbon, such as a straight or branched group of 1-12, 1-10, 1-6, 1-4, or 1-3 carbon atoms, referred to herein as C₁-C₁₂alkyl, C₁-C₁₀alkyl, C₁-C₆alkyl, C₁₋₄alkyl, and C₁₋₃alkyl respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc. In certain embodiments, alkyl refers to C₁-C₆alkyl. In certain embodiments, cycloalkyl refers to C₃-C₆cycloalkyl.

Alkyl, alkenyl and alkynyl groups can, in some embodiments, be optionally be substituted with or interrupted by at least one group selected from alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl.

The term “alkynyl” as used herein refers to an unsaturated straight or branched hydrocarbon having at least one carbon-carbon triple bond, such as a straight or branched group of 2-12, 2-8, or 2-6 carbon atoms, referred to herein as C₂-C₁₂alkynyl, C₂-C₈alkynyl, and C₂-C₆alkynyl, respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl, etc.

The term “amide” or “amido” as used herein refers to a radical of the form —RₐC(O)N(R_{b})—, —RₐC(O)N(R_{b})R_{c}—, or —C(O)NR_{b}R_{c}, wherein Rₐ, R_{b} and R_{c} are each independently selected from alkyl, alkenyl, alkynyl, aryl, arylalkyl, cycloalkyl, heteroaryl, heterocyclyl, and hydrogen. The amide can be attached to another group through the carbon, the nitrogen, R_{b}, R_{c}, or Rₐ. The amide also may be cyclic, for example R_{b} and R_{c}, Rₐ and R_{b}, or Rₐ and R_{c} may be joined to form a 3- to 12-membered ring, such as a 3- to 10-membered ring or a 5- to 6-membered ring.

The term “amidino” as used herein refers to a radical of the form —C(═NR)NR′R″ where R, R′, and R″ can each independently be selected from alkyl, alkenyl, alkynyl, aryl, arylalkyl, cycloalkyl, heteroaryl, and heterocyclyl.

The term “amine” or “amino” as used herein refers to a radical of the form —NR_{d}Rₑ, —N(R_{d})Rₑ—, or —RₑN(R_{d})R_{f}— where R_{d}, Rₑ, and R_{f} are independently selected from alkyl, alkenyl, alkynyl, aryl, arylalkyl, cycloalkyl, haloalkyl, heteroaryl, heterocyclyl, and hydrogen. The amino can be attached to the parent molecular group through the nitrogen, R_{d}, Rₑ or R_{f}. The amino also may be cyclic, for example any two of R_{d}, Rₑ or R_{f} may be joined together or with the N to form a 3- to 12-membered ring, e.g., morpholino or piperidinyl. The term amino also includes the corresponding quaternary ammonium salt of any amino group, e.g., —[N(R_{d})(Rₑ)(R_{f})]⁺. Exemplary amino groups include aminoalkyl groups, wherein at least one of R_{d}, Rₑ, or R_{f} is an alkyl group.

The term “aryl” as used herein refers to a mono- , bi- , or other multi-carbocyclic, aromatic ring system. In certain embodiments, aryl refers to a monocyclic and/or bicyclic, 6 to 10 membered ring. The aromatic ring may be substituted at one or more ring positions with substituents selected from alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, and/or aryls. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl.

The term “arylalkyl” as used herein refers to an aryl group having at least one alkyl substituent, e.g., -aryl-alkyl. Exemplary arylalkyl groups include, but are not limited to, arylalkyls having a monocyclic aromatic ring system, wherein the ring comprises 6 carbon atoms. For example, “phenylalkyl” includes phenylC₄alkyl, benzyl, 1-phenylethyl, 2-phenylethyl, etc.

The term “carbonyl” as used herein refers to the radical —C(O)—.

The term “carboxy” as used herein refers to the radical —COOH or its corresponding salts, e.g., —COONa, etc.

The term “cyano” as used herein refers to the radical —CN.

The term “cycloalkyl” as used herein refers to a monovalent saturated or unsaturated cyclic, bicyclic, or bridged bicyclic hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclohexenes, cyclopentanes, cyclopentenes, cyclobutanes and cyclopropanes. Cycloalkyl groups may be substituted with alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. Cycloalkyl groups can be fused to other cycloalkyl, aryl, or heterocyclyl groups. In certain embodiments, cycloalkyl refers to C₃-C₆ alkyl.

The terms “halo” or “halogen” as used herein refer to F, Cl, Br, or I.

The term “haloalkyl” as used herein refers to an alkyl group substituted with one or more halogen atoms.

The term “nitro” as used herein refers to the radical —NO₂.

The term “phenyl” as used herein refers to a 6-membered carbocyclic aromatic ring. The phenyl group can also be fused to a cyclohexane or cyclopentane ring. Phenyl can be substituted with one or more substituents including alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl.

The term “phosphate” as used herein refers to the radical —OP(O)(ORₐₐ)₂ or its anions. The term “phosphonate” refers to the radical —P(O)(ORₐₐ)₂ or its anions. The term “phosphinate” refers to the radical —PRₐₐ(O)(ORₐₐ) or its anion, where each Rₐₐ can be selected from, for example, alkyl, alkenyl, alkynyl, aryl, arylalkyl, cycloalkyl, hydrogen, haloalkyl, heteroaryl, and heterocyclyl.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The compounds of the disclosure may contain one or more stereogenic centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of the present disclosure. The symbol [symbol] denotes a bond that may be a single, double or triple bond as described herein. The present disclosure encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The term “substantially optically pure”, “substantially enantiomerically pure”, “optically pure” or “enantiomerically pure” as used herein when referring to a compound (e.g., a compound described herein) means that at least 95%, for example, at least 96%, at least 97%, or at least 98% of the compound has the desired stereogenic center in a given configuration. It will be appreciated that the percentage is expressed as a percentage of both enantiomers of the compound. For example, a compound of Formula VII is substantially optically pure if, based on the total of both the levorotatory and dextrorotatory enantiomers, at least 95% is (S)-(−)-3-(4-acetamidophenyl)-2-methoxypropionic acid (the levorotatory enantiomer).

The compounds disclosed herein can exist in solvated as well as unsolvated forms with pharmaceutically acceptable solvents such as water, ethanol, and the like, and it is intended that the disclosure embrace both solvated and unsolvated forms. In some embodiments, the compound is amorphous. In some embodiments, the compound is in a crystalline form. In some embodiments, the compound is a polymorph.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, fluorine and chlorine, such as ²H, ³H, ¹³C, ¹⁴c, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the e.g., Examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms, such as through hydrolysis in blood. For example, if a compound of the disclosure or a pharmaceutically acceptable salt, hydrate or solvate of the compound contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound of the disclosure contains an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(_{C1}-C6)alkyl)₂ or glycosyl (the radical resulting from the removal of a hydroxyl group of the hemiacetal form of a carbohydrate).

If a compound of the disclosure incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N—or di-N,N—(C₁-C₆)alkylamino, morpholino, piperidin-1-yl or pyrrolidin-1-yl.

The disclosure provides, at least in part, compounds represented by Formula (I), Formula (IV), Formula (V), Formula (VI), and Formula (VII), as depicted below. Also contemplated herein are pharmaceutical compositions that include a compound represented by Formula (I), Formula (IV), Formula (V), Formula (VI), and Formula (VII), and e.g., a pharmaceutically acceptable excipient and/or carrier.

### Compounds

Provided herein in part is a process for preparing a substantially optically pure compound of Formula (VII):

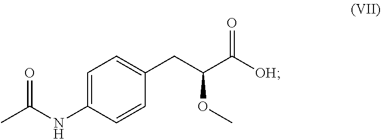 [SMILES: CO[C@@H](Cc1ccc(NC(C)=O)cc1)C(=O)O]

the process comprising:
   reacting a compound of Formula (I):

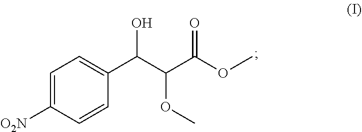 [SMILES: COC(=O)C(OC)C(O)c1ccc([N+](=O)[O-])cc1]

with an activating agent, in the optional presence of a base, to form an intermediate of Formula (I-A):

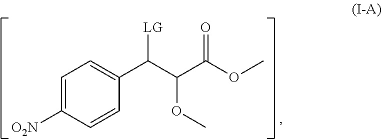

wherein LG is a leaving group;
treating the intermediate of Formula (I-A) with a base solution in the presence of an alcohol solvent, to eliminate the leaving group and thereby forming an intermediate of Formula (I-B):

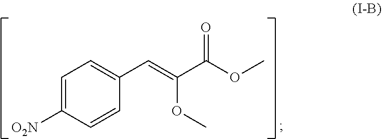

hydrolyzing the intermediate of Formula (I-B) to form a compound of Formula (IV):

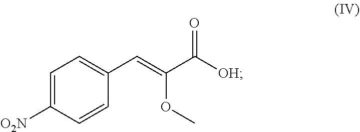 [SMILES: CO/C(=C\c1ccc([N+](=O)[O-])cc1)C(=O)O]

hydrogenating the compound of Formula (IV) to form a compound of Formula (V):

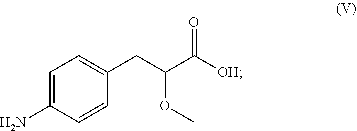

optionally resolving the compound of Formula (V) to form a substantially optically pure compound of Formula (VI):

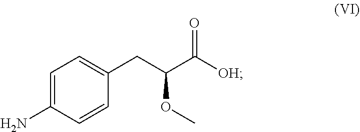

and acylating the compound of Formula (VI) to form the compound of Formula (VII).

Reacting a compound of Formula (I) with an activating agent may comprise reacting in the presence of a base and a solvent. In some embodiments, the solvent is selected from the group consisting of toluene, dichloromethane, tetrahydrofuran, diethyl ether, 2-methyltetrahydrofuran, and acetonitrile, for example, the solvent may be toluene. The base may be an amine base, for example, selected from the group consisting of triethylamine, N,N-diisopropylethylamine, and pyridine. For example, the amine base may be triethylamine.

The compound of Formula (I) can be an isolated solid prior to this step or can be dissolved in an appropriate solvent, for example, a solvent used in making or working up the compound of Formula (I). For example, the compound of Formula (I) can be unisolated and dissolved in an organic solvent, e.g., toluene, prior to this step.

Contemplated activating agent includes a sulfonylating agent, or a halogenating agent. For example, the activating agent may be selected from the group consisting of a methanesulfonyl chloride, p-toluenesulfonyl chloride, p-bromobenzenesulfonyl chloride, phenyl triflimide, triflic anhydride, and nonafluorobutanesulfonic anhydride. In some embodiments, the activating agent is methanesulfonyl chloride.

In some embodiments, the leaving group is selected from the group consisting of —OSO₂— aryl, —OSO₂-C₁₋₄alkyl, chloro, bromo, and iodo; wherein C₁₋₄alkyl and aryl may be optionally substituted with one or more substituents each independently selected, for each occurrence, from the group consisting of fluoro, bromo, and —CH₃. For example, the leaving group may be —OSO₂-phenyl or —OSO₂-C₁₋₄alkyl.

In some embodiments, the leaving group is selected from the group consisting of: —OSO₂Me,

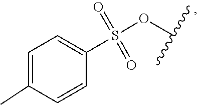 [SMILES: *OS(=O)(=O)c1ccc(C)cc1]

—OSO₂CF₃,

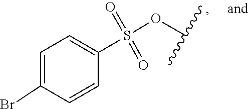 [SMILES: *OS(=O)(=O)c1ccc(Br)cc1]

—OSO₂CF₂CF₂CF₂CF₃, e.g., the leaving group may be —OSO₂Me.

Contemplated alcohol solvents may include at least one of methanol, ethanol, isopropanol, and butanol. For example, the alcohol solvent may include methanol.

In some embodiments, the base solution, comprises at least one of: sodium hydroxide, lithium hydroxide, and potassium hydroxide. For example, a base solution may include sodium hydroxide, for example, 30% sodium hydroxide.

In some embodiments, hydrolyzing the intermediate of Formula (I-B) to form a compound of Formula (IV) comprises: contacting the intermediate of Formula (I-B) with an alkali hydroxide (e.g., sodium hydroxide) and water; and neutralizing to form the compound of Formula (IV). Neutralizing can include acidifying to a pH of less than or equal to 3 by adding an acid, for example, phosphoric acid or hydrochloric acid, or a mixture thereof.

In some embodiments, hydrogenating the compound of Formula (IV) to form a compound of Formula (V) comprises contacting the compound of Formula (IV) with hydrogen and a catalyst, for example, a catalyst selected from the group consisting of PtO₂, Pd(OH)₂/C, Pt/C, 10% Pd/C, and 5% Pd/C, e.g., 5% Pd/C catalyst. Hydrogenating may be performed at a reaction temperature which is maintained between about 60-80° C. and at a pressure between about 3 to 5 atm, about 3.5 to 4.5 atm, or about 4.0 to 4.5 atm. For example, hydrogenating may be performed in the presence of one or more hydrogenation solvents selected from the group consisting of an aqueous ammonia solution, methanol, ethanol, isopropanol, N,N-dimethylformamide, tetrahydrofuran and ethyl acetate, e.g., an aqueous ammonia solution, methanol, or N,N-dimethylformamide. In some embodiments the hydrogenation solvent is methanol or a mixture of methanol and ammonia, e.g., a 30% ammonia aqueous solution.

Following hydrogenation, the compound of Formula (V) may optionally be isolated by contacting the solution with an acid, for example, acetic acid or hydrochloric acid, or a mixture thereof.

Resolving the compound of Formula (V) to form a substantially optically pure compound of Formula (VI) may include:
a) resolving a compound of Formula (V) in the presence of a chiral acid thereby forming a chiral salt of the compound of Formula (VI); and
b) neutralizing the chiral salt of the compound of Formula (VI) thereby forming the compound of Formula (VI).

In some embodiments, the chiral acid is selected from the group consisting of (S)-(+)-camphor-10-sulfonic acid, (2R,3R)-(+)-tartaric acid, (S)-(−)-malic acid, (1S)-(+)-3-bromocamphor-10-sulfonic acid, (S)-1-phenylethane sulphonic acid, dibenzoyl-L-tartaric acid, glutamic acid, (1R, 3S)-camphoric acid, (1S)-camphanic acid and (R)-(—)-mandelic acid and all other chiral acids that can lead to resolution of racemic mixture or an enantiomer thereof, e.g., (S)-(+)-camphor-10-sulfonic acid.

In some embodiments, the chiral salt of the compound of Formula (VI) is:

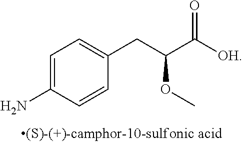

In various embodiments, resolving may further comprise adding a primer and maintaining a temperature between 30-35° C. while stirring, and/or may occur in the presence of acetone and water. In some embodiments, resolving may further comprise maintaining a temperature between 55-60° C. (e.g., 58° C.) while stirring, and/or may occur in the presence of acetone and water.

A primer may be a substantially optically pure chiral acid salt of the compound of Formula (VI). In some embodiments, the chiral acid is (S)-(+)-camphor-10-sulfonic acid.

For example, the primer may be represented by:

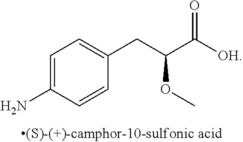

At the end of resolving, if specifications are not met, reprocessing can occur where the solution maintained at the resolving temperature while stirring for a longer time.

Neutralizing may include contacting the chiral salt of the compound of Formula (VI), with (i) an aqueous base (e.g., ammonium hydroxide); and then (ii) acidifying the solution by adding an acid (e.g., acetic acid). Neutralizing may occur in the presence of one or more solvents, e.g., in the presence of water and ethyl acetate.

Acylating may include contacting the compound of Formula (VI) with an acylating agent (e.g., acetic anhydride) in the presence of an organic solvent selected from the group consisting of ethyl acetate, tetrahydrofuran, diethyl ether, dichloromethane, and toluene, e.g., ethyl acetate. Such acylating may occur at a temperature between 60 to 70° C., for example, between 65 to 70° C. Acylating may further comprise dissolving the compound isolated from the previous step in one or more solvents, e.g., water and/or ethyl acetate, to prepare a solution and contacting the solution with an acylating agent, e.g., acetic acid. Such step may occur at a temperature between 60 to 70° C., for example, at a temperature between 65 to 70° C.

In some embodiments, after optionally resolving the compound of Formula (V) to form a substantially optically pure compound of Formula (VI), a mother liquor derived from resolution step may still contain the desired enantiomer (as a salt of the resolving agent) together with the undesired one. In these embodiments, resolving may optionally further comprise:
a) recovering the mixture of enantiomers (as chiral salts of the resolving agent) from the mother liquor, neutralizing the chiral salts to form a compound of Formula (V), and resolving the compound of Formula (V) providing additional substantially optically pure compound of Formula (VI), thereby increasing the total process yield; or
b) distilling part of the mother liquor and precipitating the desired enantiomer as a salt of the chiral resolving agent from the mother liquor, thereby increasing the total process yield.

Also contemplated herein is a process of racemizing the undesired enantiomer or chiral salt thereof in the presence of a base and resolving the resulting mixture of (R), and (S) enantiomers using a resolution process contemplated herein to form the desired enantiomer.

Contemplated bases include those selected from the group consisting of hydroxides, alkoxides (e.g., methoxide), amides (e.g., lithium diisopropylamide), hydrides (e.g., NaH), organolithiums, and Grignard reagents. For bases that require a counterion, exemplary counterions contemplated herein may include alkali metals or alkaline earth metals e.g., lithium, sodium, potassium, or calcium; or organic counterions e.g., tetraalkyl ammoniums.

In some embodiments, the compound of Formula (VII) can be produced on a multi-kilogram scale, for example, at least about 8 to 11 kg, about 13 to 15 kg, or about 130 to 150 kg is obtained. In some embodiments, at least about 130 kg of the compound of Formula (VII) is obtained.

In some embodiments, the substantially optically pure compound of Formula (VII) is at least 98% of the desired enantiomer:

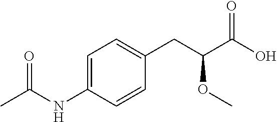 [SMILES: CO[C@@H](Cc1ccc(NC(C)=O)cc1)C(=O)O]

(expressed as a percentage of both enantiomers). In some embodiments, the content of (S)-(+3-(4-aminophenyl)-2-methoxypropionic acid may not, for example, be more than 0.15% by HPLC.

In an alternative embodiment a disclosed preparation of a substantially optically pure compound of Formula (VII) comprises acylating racemic compound (V) yielding a racemic mixture of 3-(4-acetamidophenyl)-2-methoxypropionic acid, and resolving the racemic 3-(4-acetamidophenyl)-2-methoxypropionic acid to provide substantially enantiomerically pure compound of Formula (VII). For example, forming substantially optically pure compound of Formula (VII) may comprise:
a) resolving a racemic mixture of 3-(4-acetamidophenyl)-2-methoxypropionic acid in the presence of a chiral base thereby forming a chiral salt of the compound; and
b) neutralizing the chiral salt of the compound thereby forming the compound of Formula (VII).

In some embodiments, the chiral base is selected from the group consisting of e.g., enantiomerically pure 1-amino-2-propanol, brucine, dehydroabietylamine, N,α-dimethylbenzylamine, N,N-dimethyl-1-phenylethylamine, ephedrine, α-methylbenzylamine, 1-(2-naphthyl)ethylamine, quinidine, quinine, strychnine, valine and all other chiral bases that can lead to resolution of racemic mixture. For example, a chiral salt of the compound can be represented by:

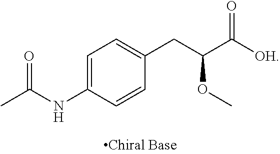 [SMILES: CO[C@@H](Cc1ccc(NC(C)=O)cc1)C(=O)O]

In certain embodiments, the present disclosure provides a process for preparing a compound of Formula (I):

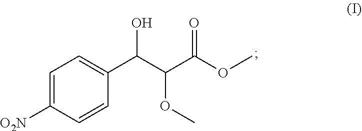 [SMILES: COC(=O)C(OC)C(O)c1ccc([N+](=O)[O-])cc1]

the process comprising, providing a mixture of a compound of Formula (II):

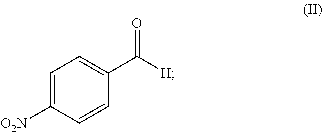

and a compound of Formula (III):

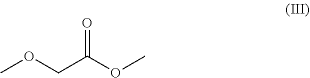

and contacting the mixture with a base (e.g., in a solvent such as tetrahydrofuran); thereby forming a compound of Formula (I).

Contacting may be performed at a temperature less than or equal to 10° C., e.g., may comprise stirring for about 5 minutes and/or is performed at a reaction temperature which is maintained between −10 to 10° C., for example, between −5 to 0° C. The base may be an alkali metal alkoxide, e.g., selected from the group consisting of sodium methoxide, lithium methoxide, and potassium methoxide, for example, sodium methoxide.

In some embodiments, provided herein is a compound represented by:

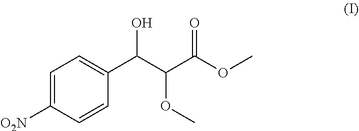 [SMILES: COC(=O)C(OC)C(O)c1ccc([N+](=O)[O-])cc1]

or a pharmaceutically acceptable salt or stereoisomer thereof.

In some embodiments, a compound of Formula (I) may exist as a mixture of stereoisomers. Contemplated stereoisomers of Formula (I) include e.g., compounds of Formula (a), Formula (b), Formula (c), or Formula (d):

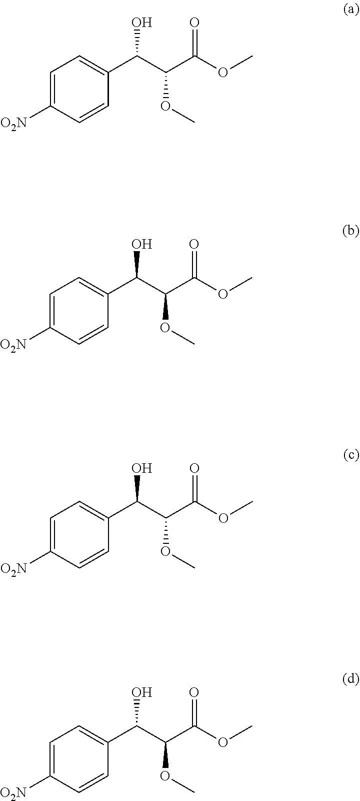 [SMILES: COC(=O)[C@H](OC)[C@@H](O)c1ccc([N+](=O)[O-])cc1 ; COC(=O)[C@@H](OC)[C@H](O)c1ccc([N+](=O)[O-])cc1 ; COC(=O)[C@H](OC)[C@H](O)c1ccc([N+](=O)[O-])cc1 ; COC(=O)[C@@H](OC)[C@@H](O)c1ccc([N+](=O)[O-])cc1]

Also provided herein in part is a process for preparing a substantially optically pure compound of Formula (VI):

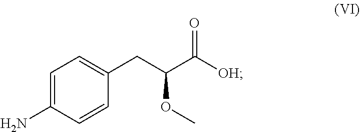

the process comprising: reacting a compound of Formula (I):

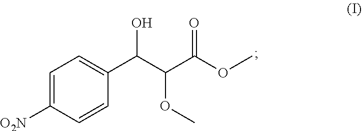 [SMILES: COC(=O)C(OC)C(O)c1ccc([N+](=O)[O-])cc1]

with an activating agent, in the optional presence of a base, to form an intermediate of Formula (I-A):

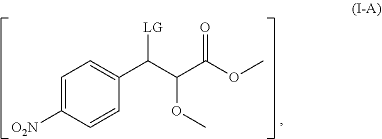

wherein LG is a leaving group;
treating the intermediate of Formula (I-A) with a base solution in the presence of an alcohol solvent, to eliminate the leaving group and thereby forming an intermediate of Formula (I-B):

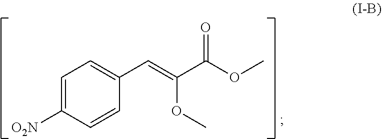

hydrolyzing the intermediate of Formula (I-B) to form a compound of Formula (IV):

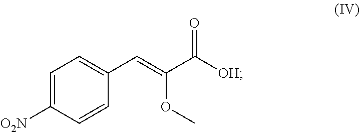 [SMILES: CO/C(=C\c1ccc([N+](=O)[O-])cc1)C(=O)O]

hydrogenating the compound of Formula (IV) to form a compound of Formula (V):

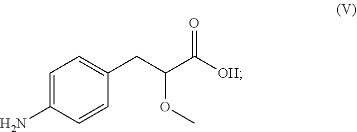

resolving the compound of Formula (V) to form a substantially optically pure compound of Formula (VI), wherein the process and variables are as defined herein.

Procedures for making compounds described herein are provided below with reference to Schemes 1-21. In the reactions described below, it may be necessary to protect reactive functional groups (such as hydroxyl, amino, or carboxyl groups) to avoid their unwanted participation in the reactions. The incorporation of such groups, and the methods required to introduce and remove them are known to those skilled in the art (for example, see Greene, Wuts, Protective Groups in Organic Synthesis. 4th Ed. (2007)). The deprotection step may be the final step in the synthesis such that the removal of protecting groups affords compounds of Formula I, as disclosed herein. Starting materials used in the following schemes can be purchased or prepared by methods described in the chemical literature, or by adaptations thereof, using methods known by those skilled in the art. The order in which the steps are performed can vary depending on the groups introduced and the reagents used, but would be apparent to those skilled in the art.

### EXAMPLES

The procedures disclosed herein can be conducted in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as intermediates e.g., as part of a synthetic scheme disclosed herein are contemplated as compounds of the disclosure

### ABBREVIATIONS

### General

APCI atmospheric pressure chemical ionization
DSC differential scanning calorimetry
EA elemental analysis
ESI electrospray ionization
GC gas chromatography
HPLC high-performance liquid chromatography
ICP-AES inductively coupled plasma atomic emission spectroscopy
LC liquid chromatography
MHz megahertz
MS mass spectrometry
NMR nuclear magnetic resonance
TLC thin layer chromatography
Me methyl
Ph phenyl
Et ethyl

### Solvents and Reagents

CSA camphorsulfonic acid
DMF N,N-dimethylformamide
EtOAc ethyl acetate
Mesyl methanesulfonyl
NaOMe sodium methoxide
THF tetrahydrofuran

### General Experimental:

¹NMR spectra were recorded using a Varian Gemini 200 NMR-spectrometer operating at 200 MHz or 600 MHz. Chemical shifts for protons were reported as parts per million in δ scale using solvent residual peak (DMSO-d₆: 2.50 ppm) as an internal standard. Data are represented as follows: chemical shift (δ), multiplicity (s=singlet, d=doublet, t=triplet, q=quartet, qn=quintuplet, sx=sextet, sp=septuplet, m=multiplet, br=broad, dd=doublet of doublets, dt=doublet of triplets, qd=quartet of doublets, dquin=doublet of quintets), coupling constant (J, Hz) and integration (#H).

¹³C NMR were recorded dissolving the sample in DMSO-d₆ operating at 600 MHz. Full decoupled spectra were acquired.

Mass spectra were recorded on an Thermo-Finnigan LCQ-Advantage mass-spectrometer or MS Thermo LCQ-fleet. The LC/MS data were obtained using positive/negative mode switching or using a negative mode polarity with acquisition parameters optimized in negative polarity on signal 236 m/z corresponding to the quasi-molecular ion [M-H]⁻ of the sample.

The elemental analysis (CHN) was carried out by Carlo Erba EA1108 equipment under the following conditions: sample weight: 0.5-2 mg; furnace temperature: 1010° C.; column temperature: 80° C.; gas: He; flow: 100 mL/min. The elemental analysis (Oxygen) was carried out by Carlo Erba EA1108 equipment under the following conditions: sample weight: 0.5-2 mg; furnace temperature: 1010° C.; column temperature: 60° C.; gas: He; flow: 100 mL/min.

FT-IR spectroscopy FT-IR/ATR was performed on the sample as such with a Perkin Elmer spectrometer model Spectrum Two instrument equipped with a diamond probe. The spectrum was collected in the frequency range of 450-4000 cm⁻¹.

UV-visible spectrum was recorded with a spectrophotometer Shimadzu UV 2600 working under the following conditions: cuvette: quartz 1 cm; range: 200-600 nm; scan speed: medium; scan step: 1 nm; slit width: 1.0 nm; Reference solution: methanol; sample solution: 1 mg of sample was dissolved in 100 mL of methanol.

DSC was performed using a Mettler-Toledo TGA-DSC1 instrument working in the following conditions (pan: aluminum (open); heating rate: 10° C./min; gas: Nitrogen; flow: 30 mL/min).

Specific optical rotation was determined as follows. In a 50 mL volumetric flask accurately weigh 0.5 g of the sample, dissolve and dilute to volume with methanol (concentration: 10 mg/mL corresponding to 1% w/v). Determine the rotation angle of the obtained solution, using a polarimeter tube having an optical length corresponding to 1 dm. Calculate the specific optical rotation [α]_{D}²⁰ referred to the dried substance with the following formula: (α×50×100)/(W×(100−m)), where α=read rotation angle; W=sample weight (g); and m=sample water content (%).

Chiral purity (HPLC) was determined as follows.

### Apparatus and Operative Conditions

| | |
|---|---|
| Chromatograph | HPLC Waters equipped with pump, injector, UV-Vis |
| | spectrophotometer and Empower integration system |
| | (or equivalent) |
| Column | DaicelChiralpak WH, 10 μm 250 mm × 4.6 mm I.D. |
| | (Daicel Chemical Industries DAIC25625) |
| Mobile phase A | dissolve 62.5 mg of CuSO₄•5H₂O in 1000 mL of |
| | water |
| Mobile phase | Prepare a 90:10 (v/v) mixture of Mobile phase A/ |
| | acetonitrile |
| Flow rate | 1.0 mL/min |
| Column | 45° C. |
| temperature | |
| Injected | 20 μl |
| volume | |
| Wavelength | 258 nm |

### Preparation of the Solutions

Sample solution: in a 10 mL volumetric flask accurately weigh 10 mg of the sample, then dissolve and dilute to volume with mobile phase (concentration: 1000 μg/mL).
Reference solution: in a 100 mL volumetric flask accurately weight 10 mg of (R,S)-3-(4-Acetamidophenyl)-2-methoxypropionic acid standard, then dissolve and dilute to volume with mobile phase.
System suitability test (SST) solution: in a 10 mL volumetric flask accurately weight 10 mg of (S)-(−)-3-(4-Acetamidophenyl)-2-methoxypropionic acid standard, add 2 mL of reference solution, then dissolve and dilute to volume with mobile phase (final concentration of the dextrorotatory enantiomer: 10 μg/mL corresponding to 1% with reference to the sample solution, corresponding to 99% of chiral purity of (S)-(−)-3-(4-Acetamidophenyl)-2-methoxypropionic acid).

### System Suitability Test and Procedure

Inject 20 μ1 of the sample solution and of SST solution in the chromatograph and record the chromatogram.

The elution order of the main peaks is as follows. (R)-(+)-3-(4-Acetamidophenyl)-2-methoxypropionic acid (dextrorotatory enantiomer): RRT=0.9; and (S)-(−)-3-(4-Acetamidophenyl)-2-methoxypropionic acid (levorotatory enantiomer): RRT=1.0.

The chromatographic system can be used for the test if the h/v ratio is not less than 1.5, where h is the distance between the top of the peak due to the dextrorotatory enantiomer and the baseline, v is the distance between the lowest point of the valley defined between dextrorotatory enantiomer peak and levorotatory enantiomer one and the baseline.

The chiral purity % is calculated from the following expression: (A₁×100)/(A₁+A_{d}), where A₁=(S)-(−)-3-(4-Acetamidophenyl)-2-methoxypropionic acid peak area in the sample solution (levorotatory enantiomer) and A_{d}=(R)-(+)-3-(4-Acetamidophenyl)-2-methoxypropionic acid peak area in the sample solution (dextrorotatory enantiomer).

The above method may be used to determine optical purity or enantiomeric purity of a compound as referenced herein.

### Example 1: Preparation of methyl 3-hydroxy-2-methoxy-3-(4-nitrophenyl)propionate (I)

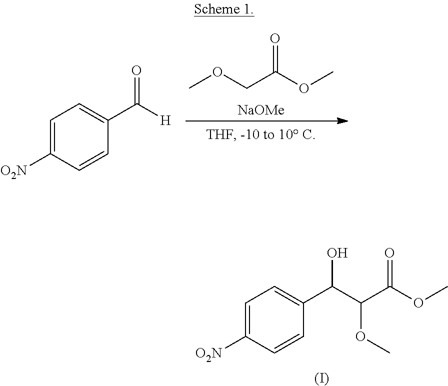

TABLE 1
Batch formula at production scale (synthesis of compound (I))

| Material | Factor* | Amount (kg) |
|---|---|---|
| sodium methoxide | 0.6 | 30.0 |
| THF | 1.5 | 75 |
| 4-nitrobenzaldehyde | 1.0 | 50 |
| methyl methoxyacetate | 1.1 | 55 |
| THF | 2.0 | 100 |
| THF | 0.5 | 25.0 |
| cold toluene | 2.0 | 100 |
| glacial acetic acid | 1.0 | 50 |
| deionized water | 3.0 | 150 |
| deionized water | 2.0 | 100 |
| sodium chloride | 0.2 | 10 |
| deionized water | 2.0 | 100 |
| sodium chloride | 0.2 | 10 |
| toluene | 1.0 | 50 |
| toluene | 1.5 | 75 |
| cold toluene | 0.5 | 25 |

*factor is referenced to the amount of 4-nitrobenzaldehyde used

### Preparation of 4-nitrobenzaldehyde in methyl methoxyacetate and tetrahydrofuran suspension

A stainless steel reactor was charged with 4-nitrobenzaldehyde (50 kg), methyl methoxyacetate (55 kg) and tetrahydrofuran (100 kg) and cooled to −5 to +5° C. while stirring.

### Preparation of methyl 3-hydroxy-2-methoxy-3-(4-nitrophenyl)propionate (I)

A stainless steel reactor was flushed with nitrogen (2×), and charged with sodium methoxide (30.0 kg) and tetrahydrofuran (75 kg) while flushing with nitrogen. The reactor was then flushed with nitrogen for 1 min and the sodium methoxide solution cooled to −10 to −5° C., while stirring. The cooled solution was then treated with the previously prepared suspension of 4-nitrobenzaldehyde in methyl methoxyacetate and tetrahydrofuran maintaining the temperature of the reaction mass below 10° C. After the addition was complete, the empty reactor was rinsed with tetrahydrofuran (25.0 kg) and poured into the reaction. The reaction was stirred at −10 to +10° C. for no more than 5 minutes. Maintaining the temperature at −10 to +10° C., cold toluene (100 kg, −10 to 0° C.) followed by glacial acetic acid (50 kg) were slowly added and the reaction stirred for 10 minutes at −10 to +10° C. Deionized water (150 kg) was added and the reaction mass stirred at 0 to 10° C. for at least 10 minutes, then at 20 to 30° C. for at least 10 minutes to ensure complete dissolution, after which time stirring was stopped and the phases allowed to separate. The aqueous phase was eliminated, and the separated organic phase was treated with aqueous sodium chloride solution (previously prepared by adding 10 kg of sodium chloride to 100 kg of deionized water). The mass was then heated to 50 to 60° C., while stirring for at least 10 minutes. Stirring was stopped and the phases allowed to separate. The aqueous phase was eliminated and the organic phase treated with sodium chloride (previously prepared by adding 10 kg of sodium chloride with 100 kg of deionized water). The mass was extracted once more using the same protocol. The separated organic phase was then distilled under vacuum, without exceeding 80° C. to remove THF. The reactor containing the hot residue was flushed with nitrogen then treated with toluene (50 kg), and the toluene distilled under vacuum, without exceeding 80° C. The resulting hot residue was flushed with nitrogen then treated with toluene (75 kg) and the mass stirred for at least 30 minutes at 30 to 45° C. to ensure good product precipitation. The mixture was then cooled to −10 to 0° C. and stirred at for at least 1 hour at that temperature. The resulting suspension was centrifuged portion-wise, washing with cold toluene (25 kg) to obtain wet methyl 3-hydroxy-2-methoxy-3-(4-nitrophenyl)propanoate (I) (84 kg), which was used directly.

Methyl 3-hydroxy-2-methoxy-3-(4-nitrophenyl)propanoate (I): IR: 3468, 1737, 1514, 1350, 1201, 1101 cm⁻¹; LCMS (−)APCI: m/z calculated for C₁₁H₁₃NO₆: 255, found: 254 (M-H); ¹H NMR (200 MHz, DMSO-d ₆): δ 8.16 (AA′BB′ system, J=8.8 Hz, 2 H), 7.62 (AA′BB′ system, J=8.8 Hz, 2 H), 5.97 (d, J=6.0 Hz, 1 H), 5.05 (dd, J=6.0, 4.0 Hz, 1 H), 4.10 (d, J=4.0 Hz, 1 H), 3.60 (s, 3 H), 3.20 (s, 3 H); ¹³C NMR (150 MHz, DMSO-d₆): δ 170.2, 149.4, 146.7, 127.8, 122.9, 84.1, 72.7, 58.2, 51.6; UV-Vis (MeOH): λₘₐₓ 202, 270; Anal. calcd for: (C₁₁H₁₃NO₆): C 51.97; H 5.16; N 5.42; O 37.52.

### Example 2: Preparation of (S)-(−)-3-(4-Aminophenyl)-2-methoxypropionic acid (VI) and (5)-(−)- 3-(4-Acetamidophenyl)-2-methoxypropionic acid (VII)

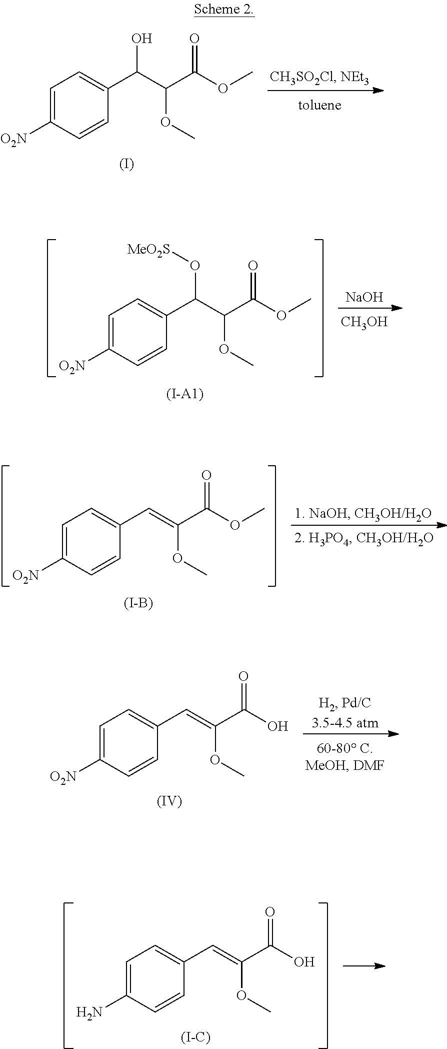

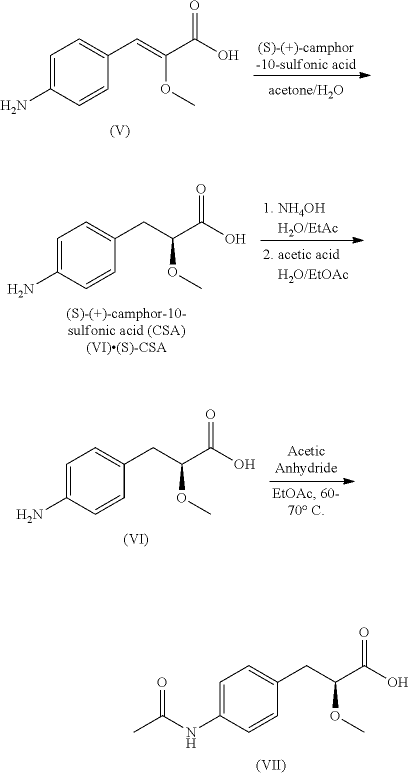

### Preparation of 2-methoxy-3-(4-nitrophenyl)acrylic acid (IV)

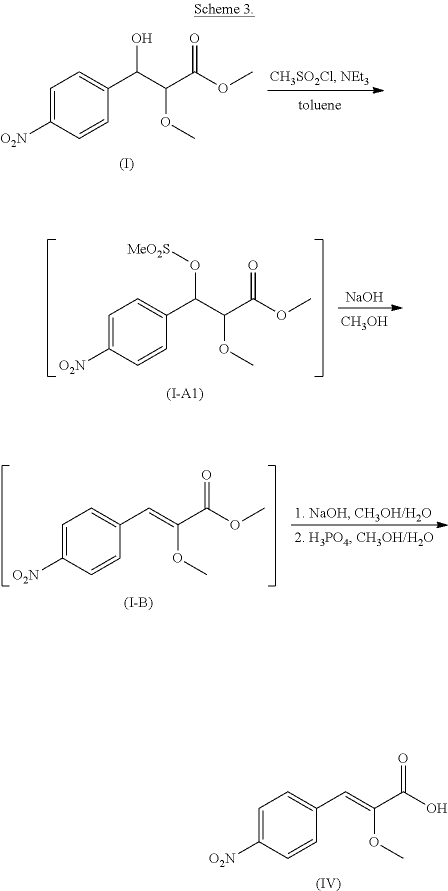

TABLE 2
Batch formula at production scale (synthesis of compound (IV))

| Material | Factor* | Amount (kg) |
|---|---|---|
| methyl 3-hydroxy-2-methoxy-3- | 1.0 | 84 (72.6 |
| (4-nitrophenyl)propanoate, wet | | kg as dried) |
| toluene | 3.9 | 283 |
| triethylamine | 0.5833 | 42.3 |
| methanesulfonyl chloride | 0.5 | 36.3 |
| deionized water | 2.0 | 145 |
| toluene | 0.1 | 7.3 |
| methanol | 3.0 | 218 |
| sodium hydroxide 30% | 1.3 | 94 |
| deionized water | 4.0 | 290 |
| phosphoric acid 85% | 1.3 | 94 |
| phosphoric acid 85% | — | to pH ≤3.0 |
| deionized water | 0.5 | 36.3 |
| toluene | 1.0 | 73 |
| deionized water | 2.0 | 145 |

*factor is referenced to the amount of methyl 3-hydroxy-2-methoxy-3-(4-nitrophenyl)propanoate (as dried) used

A stainless steel reactor flushed with nitrogen (2×) was loaded with wet methyl 3-hydroxy-2-methoxy-3-(4-nitrophenyl)propanoate (I) (84 kg) and toluene (283 kg) then cooled to 0 to 10° C. while flushing with nitrogen. Maintaining the temperature at 0 to 10° C. triethylamine (42.3 kg) and methanesulfonyl chloride (36.3 kg) were added, and the reaction stirred at 0 to 10° C. for 30 minutes. Deionized water (145 kg) was slowly added, and the resulting mass stirred at 50 to 60° C. for 10 minutes, after which time stirring was stopped and the phases were allowed to separate. The aqueous phase was eliminated and the organic phase filtered washing with toluene (7.3 kg). The toluene was removed by distillation under vacuum and without exceeding 60° C., to obtain an oily residue. The resulting residue was then treated with methanol (218 kg), transferred to another reactor, flushed with nitrogen, cooled to 20 to 30° C. and treated slowly with 30% sodium hydroxide (94 kg). The reaction was stirred at 20 to 30° C. for 2 hours. Deionized water (290 kg) was added, and the solution heated to 55 to 65° C. At this temperature, 85% phosphoric acid (94 Kg) was slowly added until a pH≤3.0 was obtained. The resulting precipitated product was stirred at 55 to 65° C. for at least 30 minutes then cooled to 25 to 30° C., and stirred for at least 30 minutes. The mixture was centrifuged washing with deionized water (36.3 kg), toluene (73 kg), and deionized water (145 kg). The product was granulated and dried at 70 to 80° C. to deliver 2-methoxy-3-(4-nitrophenyl)acrylic acid (IV) (50.4 kg).

### Methyl 2-methoxy-3-((methylsulfonyl)oxy)-3-(4-nitrophenyl)propanoate (I-Al)

LCMS(+)ESI: m/z calculated for C₁₂H₁₅NO₈S: 333; found 356 (M+Na); ¹H NMR (200 MHz, DMSO-d₆): δ 8.25 (AA′BB′ system, J=8.5 Hz, 2 H), 7.72 (AA′BB′ system, J=8.5 Hz, 2 H), 6.00 (d, J=4.0 Hz, 1 H), 4.41 (d, J=4.0 Hz, 1 H), 3.65 (s, 3 H), 3.21 (s, 3 H), 3.11 (s, 3 H).

Methyl 2-methoxy-3-(4-nitrophenyl)acrylate (I-B): LCMS (−)APCI: m/z calculated for C₁₁H₁₁NO₅: 237; found 236 M-H); ¹H NMR (200 MHz, DMSO-d₆): δ 8.20 (AA′BB′ system, J=8.8 Hz, 2 H), 7.99 (AA′BB′ system, J=8.8 Hz, 2 H), 6.98 (s, 1 H), 3.80 (s, 3 H), 3.78 (s, 3 H).

2-Methoxy-3-(4-nitrophenyl)acrylic acid (IV): LCMS (−)APCI: m/z calculated for C₁₀H₉NO₅: 223, found: 222 (M-H); 1 H NMR (200 MHz, DMSO-d6): 6 13.37 (s, 1 H), 8.21 (AA′BB' system, J=9.1 Hz, 2 H), 7.98 (AA′BB' system, J=9.1 Hz, 2 H), 6.93 (s, 1 H), 3.79 (s, 3 H).

### Preparation of 3-(4-aminophenyl)-2-methoxypropanoic acid (V):

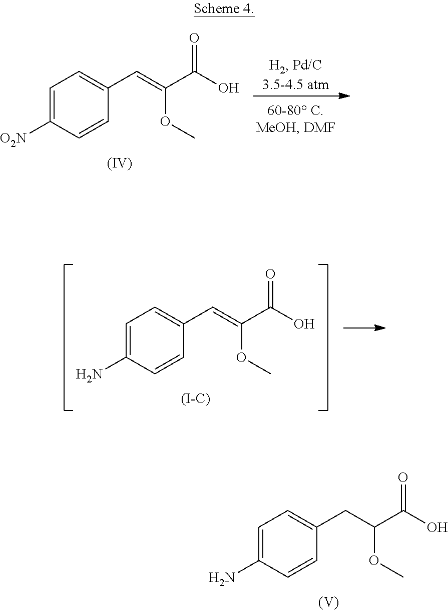

TABLE 3
Batch formula at production scale (synthesis of compound (V))

| Material | Factor* | Amount (kg) |
|---|---|---|
| 2-methoxy-3-(4- | 1.0 | 50.4 |
| nitrophenyl)acrylic | | |
| acid, dried | | |
| methanol | 5.0 | 252 |
| N,N-dimethylformamide | 0.3421 | 17.2 |
| palladium on carbon 5% | 0.1 | 7.7 |
| (containing 50% water) | | |
| N,N-dimethylformamide | 0.0526 | 2.65 |
| N,N-dimethylformamide | 0.0526 | 2.65 |
| N,N-dimethylformamide | 0.0526 | 2.65 |
| ammonia (30%) | 0.4 | 20.2 |
| deionized water | 0.25 | 12.6 |
| deionized water | 0.5 | 25.2 |
| deionized water | 3.0 | 151 |
| acetic acid 80% | 0.5 | 25.2 |
| ethyl acetate | 1.0 | 50 |
| deionized water | 2.0 | 101 |
| ethyl acetate | 0.5 | 25.2 |

*factor is referenced to the amount of dried 2-methoxy-3-(4-nitrophenyl)acrylic acid used

A suitable stainless steel reactor was loaded with N,N-dimethylformamide (17.2 kg), 5% palladium on carbon (7.7 kg) and N,N-dimethylformamide (2×2.65 kg) and the suspension stirred thoroughly.

A stainless steel reactor flushed with nitrogen (2×) charged with methanol (252 kg) was cooled to 0 to 10° C. and dry 2-methoxy-3-(4-nitrophenyl)acrylic acid (IV) (50.4 kg) was added. The resulting solution was flushed with nitrogen (2×) and treated with a previously prepared solution of 5% palladium on carbon in N,N-dimethylformamide, and the empty vessel was washed with N,N-dimethylformamide (2.65 kg). The reaction was flushed with nitrogen (2×), heated to 60 to 80° C. then charged with hydrogen until a pressure of 3.5 and 4.5 atm was obtained. The reaction was allowed to proceed, maintaining the pressure between 3.5 and 4.5 atm until hydrogen consumption ceased and the reaction was confirmed complete. The reactor was restored to atmospheric pressure and the reaction was cooled to 20 to 30° C., flushed with nitrogen (2×) and treated sequentially with 30% ammonia (20.2 kg) and deionized water (12.6 kg), stirring at 20 to 30° C. until complete dissolution occurred. The solution was filtered through sparkler filter washing with deionized water (25.2 kg). The reactor was flushed with nitrogen, and the solvents were removed by vacuum distillation, at a temperature not exceeding 70° C. The residue was treated with deionized water (151 kg), heated to 60 to 70° C. and the product precipitated by adding 80% acetic acid (25.2 kg). The mixture was stirred at 60 to 70° C. for at least 10 minutes then treated with ethyl acetate (50 kg), flushed with nitrogen and stirred at 60 to 70° C. for at least 15 minutes. The reaction mass was cooled to 10 to 20° C. and stirred for at least 30 minutes. The suspension was centrifuged portion-wise washing with deionized water (101 kg) and ethyl acetate (25.2 kg). The moist product was granulated, and dried at 60 to 70° C. to obtain 3-(4-aminophenyl)-2-methoxypropanoic acid (V) (˜37.5 kg).

3-(4-Aminophenyl)-2-methoxyacrylic acid (I-C): LCMS (+)ESI: m/z calculated for C₁₀H₁₁NO₃: 193, found 194 (M+H); ¹H NMR (200 MHz, DMSO-d₆): δ 12.00 (br s, 1 H), 7.43 (AA′BB′ system, J=8.4 Hz, 2 H), 6.76 (s, 1 H), 6.53 (AA′BB′ system, J=8.4 Hz, 2 H), 5.80 (br s, 2 H), 3.61 (s, 3 H).

3-(4-Aminophenyl)-2-methoxypropanoic acid (V): IR: 3044, 2950-2830, 2623-2064, 1618-1516, 1106 cm⁻¹; LCMS (+)ESI: m/z calculated for C₁₀H₁₃NO₃: 195, found 196 (M+H); 1 H NMR (200 MHz, DMSO-d₆): δ 8.40-6.40 (br s, 3 H), 6.83 (AA′BB′ system, J=8.1 Hz, 2 H), 6.41 (AA′BB′ system, J=8.1 Hz, 2 H), 3.77 (ABX system, J=7.5, 5.3 Hz, 1 H), 3.19 (s, 3 H), 2.74 (ABX system, J=13.9, 5.3 Hz, 1 H), 2.65 (ABX system, J=13.9, 7.5 Hz, 1 H). Anal. calcd for: (C₁₀H₁₃NO₃): C 61.40; H 6.81; N 7.11; O 24.91.

### Preparation of (S)-(−)-3-(4-Aminophenyl)-2-methoxypropionic acid S-(+)-camphor-10- sulfonic acid salt

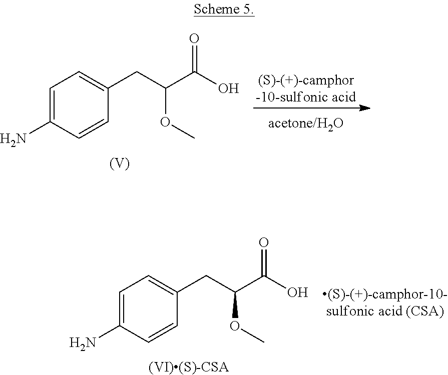

TABLE 4
Batch formula at production scale (synthesis
of compound (VI)•(S)-CSA)

| Material | Factor* | Amount (kg) |
|---|---|---|
| 3-(4-aminophenyl)-2- | 1.0 | 37.5 |
| methoxypropanoic acid | | |
| acetone | 3.0 | 113 |
| deionized water | 0.4 | 15.0 |
| (S)-(+)-10-camphor-sulfonic acid | 1.2321 | 46.2 |
| acetone | 2.0 | 75 |
| (S)-(−)-3-(4-Aminophenyl)-2- | 0.01 | 0.38 |
| methoxypropionic acid S-(+)- | | |
| camphor-10-sulfonic acid salt, | | |
| dried (primer) | | |
| acetone | 2.0 | 75 |

*factor is referenced to the amount of 3-(4-aminophenyl)-2-methoxypropanoic acid (dried) used

A stainless steel reactor was loaded with 3-(4-aminophenyl)-2-methoxypropanoic acid (V) (37.5 kg), and acetone (113 kg) while flushing with nitrogen. Deionized water (15.0 kg) and (S)-(+)-camphor-10-sulfonic acid (46.2 kg) were added and the reactor flushed with nitrogen. The reaction was heated to 45 to 55° C. until dissolution was complete. Acetone (75 kg) was added and the solution cooled to 30 to 35° C. and treated with primer (S)-(−)-3-(4-aminophenyl)-2-methoxypropionic acid S-(+)-camphor-10-sulfonic acid salt (VI)⋅(S)-CSA (0.38 kg). The reaction was stirred at 30 to 35° C. for at least 3 hours until good precipitation occurred. The suspension was centrifuged portion-wise washing with acetone (75 kg). The centrifuged product (S)-(−)-3-(4-aminophenyl)-2-methoxypropionic acid S-(+)-camphor-10-sulfonic acid salt (VI)⋅(S)-CSA (41.7 kg) was used directly in the next step.

Preparation of (S)-(−)-3-(4-Aminophenyl)-2-methoxypropionic acid (VI):

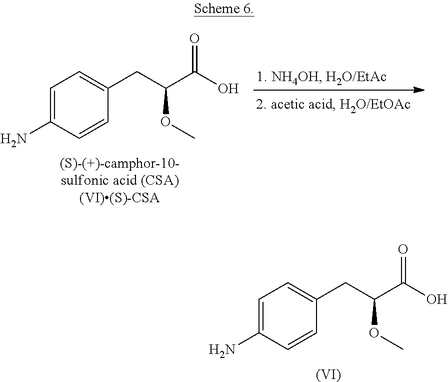

TABLE 5
Batch formula at production scale (synthesis of compound (VI))

| Material | Factor* | Amount (kg) |
|---|---|---|
| (S)-(−)-3-(4-Aminophenyl)-2- | 1.0 | 41.7 (35.0 |
| methoxypropionic acid S-(+)- | | kg as dried) |
| camphor-10-sulfonic acid salt, | | |
| wet | | |
| deionized water | 2.8 | 98 |
| deionized water | 0.2 | 7.0 |
| ethyl acetate | 1.0 | 35.0 |
| ammonia 30% | 0.1792 | 6.3 |
| acetic acid 80% | 0.125 | 4.38 |
| deionized water | 0.5 | 17.5 |
| ethyl acetate | 0.5 | 17.5 |
| deionized water | 0.5 | 17.5 |

*factor is referenced to the amount of (S)-(−)-3-(4-aminophenyl)-2-methoxypropionic acid S-(+)-camphor-10-sulfonic acid salt used (as dried).

### Preparation of (S)-(−)-3-(4-Aminophenyl)-2-methoxypropionic acid (VI)

A stainless steel reactor was charged with deionized water (98 kg) and wet ((S)-(−)-3-(4-aminophenyl)-2-methoxypropionic acid S-(+)-camphor-10-sulfonic acid salt (VI)⋅(S)-CSA (41.7 kg), flushed with nitrogen and heated for at least 10 minutes at 20 to 30° C. until complete dissolution occurs. The solution was filtered washing with deionized water (7.0 kg), then ethyl acetate (35.0 kg). The resulting solution was flushed with nitrogen, heated to 50 to 60° C. and 30% ammonia (6.3 kg) was added to precipitate the product. The resulting mixture was stirred for at least 5 minutes at 50-60° C. then 80% acetic acid (4.38 kg) was added through a filter and the reaction flushed with nitrogen and stirred at 50 to 60° C. for at least 30 minutes then cooled down to 10-20° C. and stirred for at least 1 hour. The reaction was centrifuged portion-wise and washed with deionized water (17.5 kg), ethyl acetate (17.5 kg) and deionized water (17.5 kg), and dried at 50-60° C. to deliver (S)-(−)3-(4-aminophenyl)-2-methoxypropionic acid (VI) (9.6 kg, 27%), which was used directly in the next step.

(S)-(−)-3-(4-Aminophenyl)-2-methoxypropionic acid (VI): [α]_{D}²⁰ -30 to −26 (1% w/v H₂O/CH₃OH (1:1 v/v)); IR: 2931, 2891, 2826, 2625, 2136, 1587, 1548, 1512, 1106, 1092 cm⁻¹; LCMS (+) ESI: m/z calculated for C₁₀H₁₃NO₃: 195, found 196 (M+H); ¹H NMR (200 MHz, DMSO-d₆): δ 9.0-6.0 (br s, 3 H), 6.83 (AA′BB′ system, J=8.1 Hz, 2 H), 6.43 (AA′BB′ system, J=8.1 Hz, 2 H), 3.76 (ABX system, J=7.5, 5.3 Hz, 1 H), 3.19 (s, 3 H), 2.75 (ABX system, J=13.9, 5.3 Hz, 1 H), 2.65 (ABX system, J=13.9, 7.5 Hz, 1 H). Anal. calcd for: (C₁₀H₁₃NO₃): C, 61.45; H, 6.79; N 7.12; O 24.65.

### Preparation of (S)-(−)-3-(4-Acetamidophenyl)-2-methoxypropionic acid (VII):

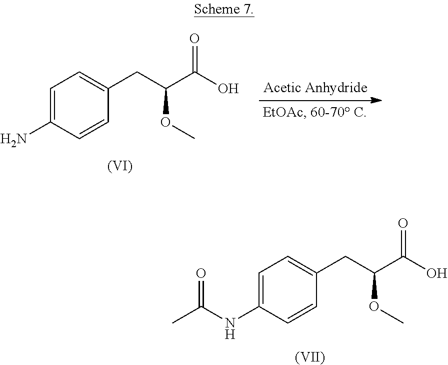

TABLE 6
Batch formula at production scale (synthesis of compound (VII))

| Material | Factor* | Amount (kg) |
|---|---|---|
| (S)-(−)-3-(4-Aminophenyl)-2- | 1.0 | 9.6 |
| methoxypropionic acid | | |
| ethyl acetate | 1.5 | 14.4 |
| acetic anhydride | 0.5833 | 5.6 |
| deionized water | 0.05 | 0.5 |
| ethyl acetate | 0.5 | 4.80 |
| deionized water | 1.5 | 14.4 |

*factor is referenced to the amount of (S)-(−)-3-(4-Aminophenyl)-2-methoxypropionic acid used

A stainless steel reactor was charged with dry (S)-3-(4-aminophenyl)-2-methoxypropanoic acid (VI) (9.6 kg) and ethyl acetate (14.4 kg) at 0 to 10° C., while flushing with nitrogen. The reaction mass was heated to 60 to 70° C. and acetic anhydride (5.6 kg) was added through a cartridge filter over about 30 minutes. The reaction was stirred at 60 to 70° C. for 1 hour, then treated with deionized water (0.5 kg) and stirred at 60 to 70° C. for at least 15 minutes during which time a precipitate formed. The mixture was cooled to 10 to 20° C. and stirred for at least 30 minutes, then centrifuged and the resulting solid washed with ethyl acetate (4.80 kg), and deionized water (14.4 kg). The resulting product was dried at 60 to 70° C. for 13-16 hours, and milled to obtain (S)-3-(4-acetamidophenyl)-2-methoxypropanoic acid (VII) (10.3 kg).

(S)-(−)-3-(4-Acetamidophenyl)-2-methoxypropionic acid: (VII): [α]_{D}²⁰-26 to −19 (1% w/v H₂O/CH₃OH (1:1 v/v)); IR: 3322, 3089, 2930, 2827, 2714, 2490, 1722, 1637, 1601, 1551, 1516, 1231, 1207, 1120, 1110 cm⁻¹; LCMS (+)ESI: m/z calculated for C₁₂H₁₅NO₄: 237, found 238 (M+H); ¹H NMR (200 MHz, DMSO-d₆): δ 12.96 (s, 1 H), 9.84 (s, 1 H), 7.43 (AA′BB′ system, J=8.5 Hz, 2 H), 7.10 (AA′BB′ system, J=8.5 Hz, 2 H), 3.86 (ABX system, J=7.6, 5.2 Hz, 1 H), 3.00 (s, 3 H), 2.95-2.70 (ABX system, J=13.9, 7.6, 5.2 Hz, 2 H), 2.00 (s, 3 H).

TABLE 7
(S)-3-(4-acetamidophenyl)-2-methoxypropanoic acid
(VII) specifications as prepared by Scheme 7

| | |
|---|---|
| (S)-(+)-camphor-10-sulfonic acid | No more than 0.1% (HPLC) |
| (S)-3-(4-aminophenyl)-2- | No more than 0.15% (HPLC) |
| methoxypropanoic acid | |
| Other impurity | No more than 0.1% (HPLC) |
| Palladium | No more than 10 ppm (ICP-AES) |
| Chiral purity | Not less than 98% (HPLC) |
| Acetic acid | No more than 5000 ppm (GC) |
| Residual solvents (ethyl acetate) | No more than 5000 ppm (GC) |

### Example 3: Preparation of methyl 3-hydroxy-2-methoxy-3-(4-nitrophenyl)propionate (I):

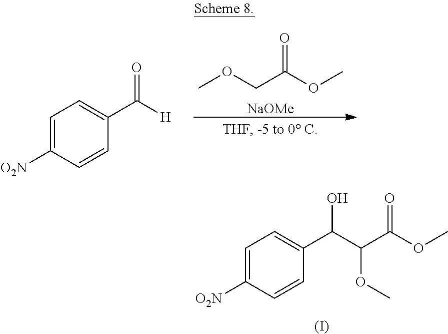

TABLE 8
Batch formula at production scale (synthesis of compound (I))

| Material | Factor* | Amount (kg) |
|---|---|---|
| sodium methoxide | 0.6 | 28.5 |
| THF | 2.5 | 119 |
| 4-nitrobenzaldehyde | 1.0 | 47.5 |
| methyl | 1.1 | 52 |
| THF | 1.0 | 47.5 |
| THF | 0.5 | 23.8 |
| cold toluene | 2.0 | 95 |
| glacial acetic acid | 0.75 | 36 |
| deionized water | 3.0 | 143 |
| deionized water | 1.0 | 48 |
| sodium chloride | 0.1 | 4.75 |
| toluene | 1.0 | 47.5 |
| toluene | 4.65 | 221 |

*factor is referenced to the amount of 4-nitrobenzaldehyde used

### Preparation of 4-nitrobenzaldehyde in methyl methoxyacetate and tetrahydrofuran suspension

A stainless steel reactor was charged with 4-nitrobenzaldehyde (47.5 kg), methyl methoxyacetate (52 kg) and tetrahydrofuran (47.5 kg) and cooled to −10 to −5° C. while stirring.

### Preparation of methyl 3-hydroxy-2-methoxy-3-(4-nitrophenyl)propionate (I)

A stainless steel reactor was flushed with nitrogen (2×), and charged with sodium methoxide (28.5 kg) and tetrahydrofuran (119 kg) while flushing with nitrogen. The reactor was then flushed with nitrogen for 1 min and the sodium methoxide solution was cooled to −10 to −7° C., while stirring. The cooled solution was then treated with the previously prepared suspension of 4-nitrobenzaldehyde in methyl methoxyacetate and tetrahydrofuran while maintaining the temperature of the reaction mass below 0° C. After the addition was complete, the empty reactor was rinsed with tetrahydrofuran (23.8 kg) and poured into the reaction. The reaction was stirred at −5 to 0° C. for no more than 5 minutes. Maintaining the temperature at −10 to 0° C., cold toluene (95 kg, −10 to 0° C.) was quickly added and then, after, glacial acetic acid (36 kg) was quickly added and the reaction was stirred for 10 minutes at-10 to +10° C. Deionized water (143 kg) was added and the reaction mass was stirred at 0 to 10° C. for at least 10 minutes, then at 25 to 30° C. for at least 10 minutes to ensure complete dissolution, after which time stirring was stopped and the phases were allowed to separate. The aqueous phase was eliminated, and the separated organic phase was treated with aqueous sodium chloride solution (previously prepared by adding 4.75 kg of sodium chloride to 48 kg of deionized water). The mass was then heated to 25 to 30° C., while stirring for at least 15 minutes. Stirring was stopped and the phases were allowed to separate. The aqueous phase was eliminated (64 kg). The separated organic phase was then distilled up to oily residue under vacuum, up to a temperature of 70 to 80° C. The reactor containing the hot residue was flushed with nitrogen then treated with toluene (47.5 kg), and the toluene was distilled under vacuum, up to a temperature of 70 to 80° C., obtaining an oily residue. The resulting hot residue was flushed with nitrogen then treated with toluene (221 kg) and the mass was stirred for at least 10 minutes at 40 to 50° C. The solution was employed as is in the subsequent step.

### Example 4: Process for the Preparation of (S)-(−)-3-(4-Aminophenyl)-2-methoxypropionic acid (VI) and (S)-(−)-3-(4-Acetamidophenyl)-2-methoxypropionic acid (VII)

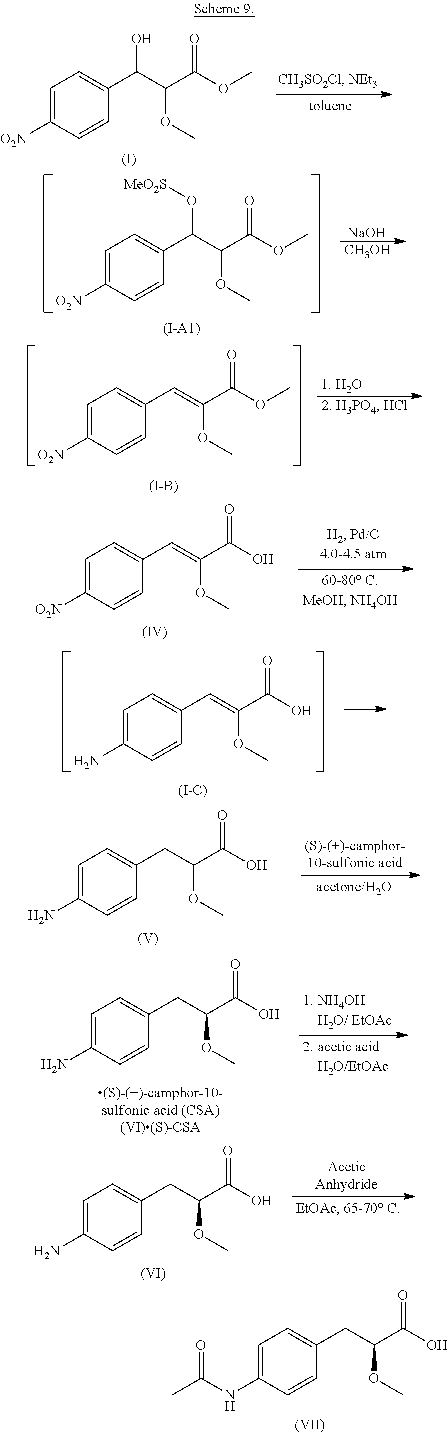

### Preparation of 2-methoxy-3-(4-nitrophenyl)acrylic acid (IV)

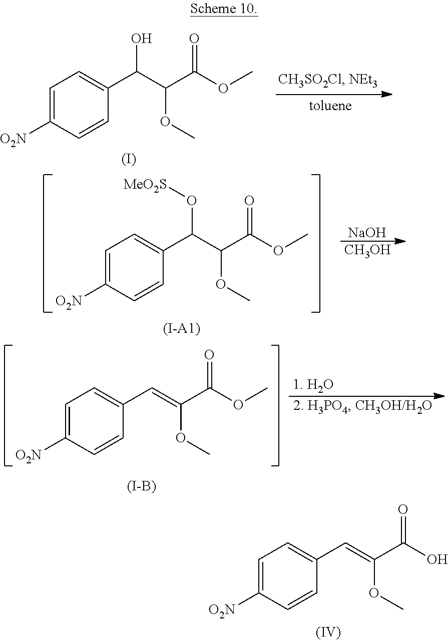

TABLE 9
Batch formula at production scale (synthesis of compound (IV))

| Material | Factor* | Amount (kg) |
|---|---|---|
| methyl 3-hydroxy-2-methoxy-3- | 1.0 | All solution obtained |
| (4-nitrophenyl)propanoate, | | in Scheme 8 |
| solution in toluene | | |
| triethylamine | 0.9 | 42.8 |
| methanesulfonyl chloride | 0.8 | 38 |
| deionized water | 3.0 | 143 |
| Toluene | 0.15 | 7.1 |
| methanol | 3.1 | 147 |
| sodium hydroxide 30% | 1.86 | 88 |
| deionized water | 3.88 | 184 |
| hydrochloric acid 37% | 0.74 | 35.2 |
| phosphoric acid 85% | 0.16 | 7.6 |
| phosphoric acid 85% | — | to pH ≤3.0 |
| deionized water | 4.0 | 190 |
| toluene | 2.4 | 114 |

*factor is referenced to the amount of 4-nitrobenzaldehyde used

The methyl 3-hydroxy-2-methoxy-3-(4-nitrophenyl)propanoate (I) in toluene solution in a stainless steel reactor was cooled to 0 to 10° C. while flushing with nitrogen, and while maintaining the temperature at 0 to 10° C. triethylamine (42.8 kg) and methanesulfonyl chloride (38 kg) were added, and the reaction was stirred at 0 to 10° C. for 60 minutes. Deionized water (143 kg) was slowly added, and the resulting mass was stirred at 55 to 60° C. for 10 minutes, after which time stirring was stopped and the phases were allowed to separate. The aqueous phase was eliminated (199 kg) and the organic phase was filtered washing the filter with toluene (7.1 kg). The filtered solution was heated to 55 to 60° C. for 10 minutes. Stirring was stopped and the phases were allowed to separate; the possible aqueous phase was sent to the waste (3.5 kg). Under vacuum and under stirring an aliquot of the toluene was distilled taking into consideration the following proportion: for 50 kg of nitrobenzaldehyde used in Scheme 8, approximately 5 kg of toluene was distilled and this aliquot of distilled toluene was sent to waste. The toluene was removed by distillation under vacuum and without exceeding 60° C., to obtain an oily residue. The resulting residue was then treated with methanol (147 kg), flushed with nitrogen, cooled to 20 to 30° C. and treated slowly with 30% sodium hydroxide (88 kg). The reaction was stirred at 20 to 30° C. for 3 hours. Deionized water (184 kg) was added, and the solution heated to 60 to 65° C. At this temperature, 37% hydrochloric acid (35.2 kg) and 85% phosphoric acid (7.6 kg) were slowly added to precipitate the product. If necessary, 85% phosphoric acid was added to obtain pH≤3.0. The resulting precipitated product was stirred at 60 to 65° C. for at least 30 minutes then cooled to 35 to 40° C., and stirred for at least 30 minutes. The mixture was centrifuged washing with deionized water (190 kg) and then washed with toluene (114 kg), (mother liquor: 749 kg). The wet product (about 82 kg) was used in the next step.

### Preparation of 3-(4-aminophenyl)-2-methoxypropanoic acid (V)

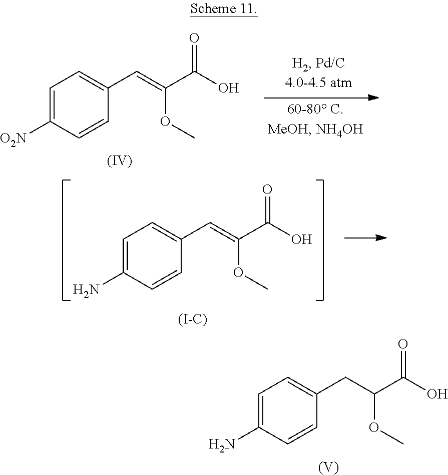

TABLE 10
Batch formula at production scale (synthesis of compound (V))

| Material | Factor* | Amount |
|---|---|---|
| 2-methoxy-3-(4-nitrophenyl)acrylic | 1.0 | 82 |
| acid, wet | | |
| methanol | 3.0 | 143 |
| Ammonia 30% | 0.3 | 14.3 |
| deionized water | 0.2 | 9.5 |
| palladium on carbon 5% (containing | 0.1 | 4.75 |
| 50% water) or palladium on carbon | | |
| 5% (recycled) | | |
| deionized water | 0.05 | 2.4 |
| deionized water | 0.05 | 2.4 |
| deionized water | 0.05 | 2.4 |
| deionized water | 0.4 | 19 |
| deionized water | 2.0 | 95 |
| hydrochloric acid 37% | 0.32 | 15.2 |
| acetic acid 80% | 0.22 | 10.5 |
| ethyl acetate | 1.5 | 71 |
| deionized water | 1.0 | 47.5 |
| ethyl acetate | 1.0 | 47.5 |

*factor is referenced to the amount of 4-nitrobenzaldehyde used

### Preparation of Aqueous Suspension of Palladium at 5% on Carbon in Water

A suitable stainless steel reactor was loaded with deionized water (9.5 kg), 5% palladium on carbon (4.75 kg) and then the bag was washed twice with deionized water (2×2.4 kg).

### Preparation of 3-(4-aminophenyl)-2-methoxypropanoic acid (V)

A stainless steel reactor flushed with nitrogen (2×) was loaded with and wet 2-methoxy-3-(4-nitrophenyl)acrylic acid (IV) (82 kg), methanol (143 kg) and 30% ammonia (14.3 kg). After restoring the atmospheric pressure, the mixture was stirred at 20 to 30° C. up to complete dissolution. The resulting solution was flushed with nitrogen (2×) and treated with a previously prepared solution of 5% palladium on carbon in deionized water, and the empty vessel was washed with deionized water (2.4 kg). The reaction was flushed with nitrogen (2×), heated to 60 to 80° C. then charged with hydrogen until a pressure of 4.0 to 4.5 atm was obtained. The reaction was allowed to proceed, maintaining the pressure between 4.0 and 4.5 atm until hydrogen consumption ceased and the reaction was confirmed complete. The reactor was restored to atmospheric pressure and the reaction was cooled to 20 to 30° C., filtered through sparkler filter, flushed with nitrogen (2×) and treated with deionized water (19 kg) that was combined with the solution containing the product. Without exceeding 50° C., the solvent was distilled under stirring and under vacuum up to oily residue. The residue was treated with deionized water (95 kg), heated to 65 to 70° C. and the product precipitated by adding 37% hydrochloric acid (15.2 kg) and 80% acetic acid (10.5 kg). The mixture was stirred at 65 to 70° C. for at least 10 minutes, and then the mixture was treated with ethyl acetate (71 kg), flushed with nitrogen and stirred at 65 to 70° C. for at least 15 minutes. The reaction mass was cooled to 15 to 20° C. and stirred for at least 30 minutes. The suspension was centrifuged portion-wise washing with deionized water (47.5 kg) and ethyl acetate (47.5 kg). The moist product was dried at 60 to 70° C. to obtain 3-(4-aminophenyl)-2-methoxypropanoic acid (V) (40.5 kg).

### Preparation of (S)-(−)-3-(4-Aminophenyl)-2-methoxypropionic acid S(+)-camphor-10-sulfonic acid salt

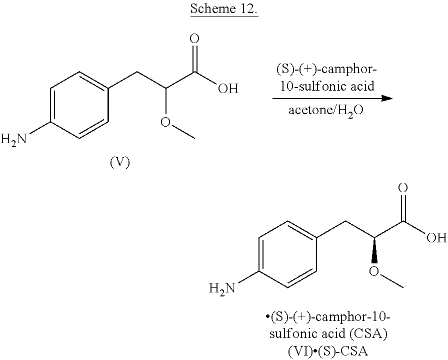

TABLE 11
Batch formula at production scale (synthesis
of compound (VI)•(S)-CSA)

| Material | Factor* | Amount (kg) |
|---|---|---|
| 3-(4-aminophenyl)-2- | 1.0 | 40.5 |
| methoxypropanoic acid | | |
| (S)-(+)-10-camphor-sulfonic acid | 1.2321 | 49.9 |
| acetone | 2.0 | 81 |
| deionized water | 0.16 | 6.5 |
| acetone | 2.0 | 81 |
| acetone | 1.5 | 61 |

*factor is referenced to the amount of 3-(4-aminophenyl)-2-methoxypropanoic acid (dried) used

A stainless steel reactor was loaded with 3-(4-aminophenyl)-2-methoxypropanoic acid (V) (40.5 kg) and (S)-(+)-camphor-10-sulfonic acid (49.9 kg); nitrogen was flushed and acetone (81 kg) was added. Deionized water (6.5 kg) was added. The reaction was heated to approximately 58° C. for 2 hours (reflux, dissolution does not occur). Acetone (81 kg) was added (temperature approximately 58° C.) and the reaction mass was kept at reflux (approx. 58° C.) for 1 hour. The reaction mass was then cooled to 37 to 42° C. and was centrifuged portion-wise washing with acetone (61 kg). The centrifuged product (S)-(−)-3-(4-aminophenyl)-2-methoxypropionic acid S-(+)-camphor-10-sulfonic acid salt (VI)⋅(S)-CSA (wet weight: 43.1 kg, 36.1 kg as dried) was used directly in the next step.

### Preparation of (S)-(−)-3-(4-Aminophenyl)-2-methoxypropionic acid (VI)

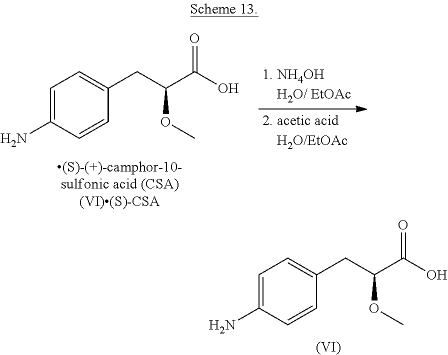

TABLE 12
Batch formula at production scale (synthesis of compound (VI))

| Material | Factor* | Amount (kg) |
|---|---|---|
| deionized water | 2.0 | 72 |
| (S)-(−)-3-(4-Aminophenyl)-2- | 1.0 | 43.1 (36.1 |
| methoxypropionic acid S-(+)- | | kg as dried) |
| camphor-10-sulfonic acid salt, | | |
| wet | | |
| deionized water | 0.2 | 7.2 |
| ethyl acetate | 1.0 | 36.1 |
| ammonia 30% | 0.1257 | 4.54 |
| acetic acid 80% | 0.05 | 1.81 |
| deionized water | 0.7 | 25.3 |
| ethyl acetate | 0.7 | 25.3 |

*factor is referenced to the amount of (S)-(−)-3-(4-aminophenyl)-2-methoxypropionic acid S-(+)-camphor-10-sulfonic acid salt used (as dried).

A stainless steel reactor was charged with deionized water (72 kg) and wet ((S)-(−)-3-(4-aminophenyl)-2-methoxypropionic acid S-(+)-camphor-10-sulfonic acid salt (VI)⋅(S)-CSA (wet weight: 43.1 kg, 36.1 kg as dried), flushed with nitrogen and heated for at least 10 minutes at 20 to 40° C. until complete dissolution occurred. The solution was filtered and washed with deionized water (7.2 kg), then ethyl acetate (36.1 kg). The resulting solution was flushed with nitrogen, heated to 55 to 60° C. and 30% ammonia (4.54 kg) was added to precipitate the product. The resulting mixture was stirred for at least 15 minutes at 55 to 60° C. then 80% acetic acid (1.81 kg) was added through a filter and the reaction flushed with nitrogen and stirred at 55 to 60° C. for at least 30 minutes then cooled down to 2-7° C. and stirred for at least 1 hour. The reaction mass was centrifuged portion-wise and washed with deionized water (25.3 kg), ethyl acetate (25.3 kg) to deliver wet (S)-(−)-3-(4-aminophenyl)-2-methoxypropionic acid (VI) (wet weight 11.8 kg, 10.8 kg as dried), which was used (wet) directly in the next step.

### Preparation of (S)-(−)-3-(4-Acetamidophenyl)-2-methoxypropionic acid (VII)

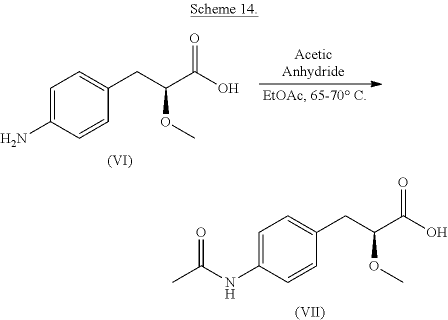

TABLE 13
Batch formula at production scale (synthesis of compound (VII))

| Material | Factor* | Amount (kg) |
|---|---|---|
| Wet (S)-(−)-3-( 4-Aminophenyl)- | 1.0 | 11.8 (10.8 |
| 2- methoxypropionic acid | | kg as dried) |
| ethyl acetate | 1.5 | 16.2 |
| acetic anhydride | 0.65 | 7.0 |
| ethyl acetate | 0.5 | 5.4 |
| deionized water | 1.5 | 16.2 |

*factor is referenced to the amount of (S)-(−)-3-(4-aminophenyl)-2-methoxypropionic acid used (as dried)

A stainless steel reactor was charged with wet (S)-3-(4-aminophenyl)-2-methoxypropanoic acid (VI) (wet weight 11.8 kg, 10.8 kg as dried) and ethyl acetate (16.2 kg), under nitrogen. The mixture was heated to 65 to 70° C. and acetic anhydride (7.0 kg) was added through a cartridge filter over about 15 minutes. The reaction was stirred at 65 to 70° C. for 60 minutes and, once the reaction end was detected, continuously stirred until formation of a precipitate. The mixture was cooled to 10 to 20° C. and stirred at 10 to 20° C. for 30 minutes, then centrifuged and the resulting solid washed with ethyl acetate (5.4 kg), and deionized water (16.2 kg). The resulting product was dried at 60 to 70° C. for 13-16 hours, and milled to obtain (S)-3-(4-acetamidophenyl)-2-methoxypropanoic acid (VII) (12.3 kg).

### Example 5: Preparation of methyl 3-hydroxy-2-methoxy-3-(4-nitrophenyl)propionate (I)

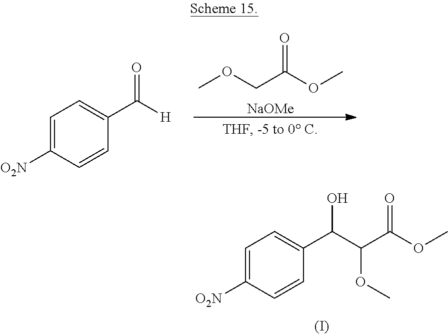

TABLE 14
Batch formula at production scale (synthesis of compound (I))

| Material | Factor* | Amount (kg) |
|---|---|---|
| sodium methoxide | 0.6 | 228 |
| THF | 2.895 | 1100 |
| 4-nitrobenzaldehyde | 1.0 | 380 |
| methyl | 1.1 | 418 |
| THF | 1.0 | 380 |
| THF | 0.105 | 40 |
| cold toluene | 2.0 | 760 |
| glacial acetic acid | 0.75 | 285 |
| deionized water | 3.0 | 1140 |
| deionized water | 1.0 | 380 |
| sodium chloride | 0.1 | 38 |
| toluene | 1.0 | 380 |
| toluene | 4.65 | 1767 |

*factor is referenced to the amount of 4-nitrobenzaldehyde used

### Preparation of 4-nitrobenzaldehyde in methyl methoxyacetate and tetrahydrofuran suspension

A stainless steel reactor was charged with 4-nitrobenzaldehyde (380 kg), methyl methoxyacetate (418 kg) and tetrahydrofuran (380 kg) and cooled to −10 to −5° C. while stirring.

### Preparation of methyl 3-hydroxy-2-methoxy-3-(4-nitrophenyl)propionate (I)

A stainless steel reactor was flushed with nitrogen (2×), and charged with sodium methoxide (228 kg) and tetrahydrofuran (1100 kg) while flushing with nitrogen. The reactor was then flushed with nitrogen for 1 min and the sodium methoxide solution cooled to −15 to −10° C., while stirring. The cooled solution was then treated with the previously prepared suspension of 4-nitrobenzaldehyde in methyl methoxyacetate and tetrahydrofuran while maintaining the temperature of the reaction mass below 0° C. After the addition was complete, the empty reactor was rinsed with tetrahydrofuran (40 kg) and poured into the reaction. The reaction was stirred at −5 to 0° C. for no more than 5 minutes. Maintaining the temperature at −10 to 0° C., cold toluene (760 kg, −10 to 0° C.) and, at the same time, glacial acetic acid (285 kg) were quickly added and the reaction stirred for 10 minutes at −10 to +10° C. Deionized water (1140 kg) was added and the reaction mass stirred at 0 to 10° C. for at least 10 minutes, then at 25 to 30° C. for at least 10 minutes to ensure complete dissolution, after which time stirring was stopped and the phases were allowed to separate. The aqueous phase was eliminated, and the separated organic phase was treated with aqueous sodium chloride solution (previously prepared by adding 38 kg of sodium chloride to 380 kg of deionized water). The mass was then heated to 25 to 30° C., while stirring for at least 10 minutes. Stirring was stopped and the phases allowed to separate. The aqueous phase was eliminated. The separated organic phase was then distilled up to oily residue under vacuum, up to a temperature of 70 to 80° C. The reactor containing the hot residue was flushed with nitrogen then treated with toluene (380 kg), and the toluene distilled under vacuum, up to a temperature of 70 to 80° C., obtaining an oily residue. The resulting hot residue was flushed with nitrogen then treated with toluene (1767 kg) and the mass stirred for at least 10 minutes at 40 to 50° C. The solution was employed as is in the subsequent step.

### Example 6: Process for the Preparation of (S)-(−)-3-(4-Aminophenyl)-2-methoxypropionic acid (VI) and (S)-(−)-3-(4-Acetamidophenyl)-2-methoxypropionic acid (VII)

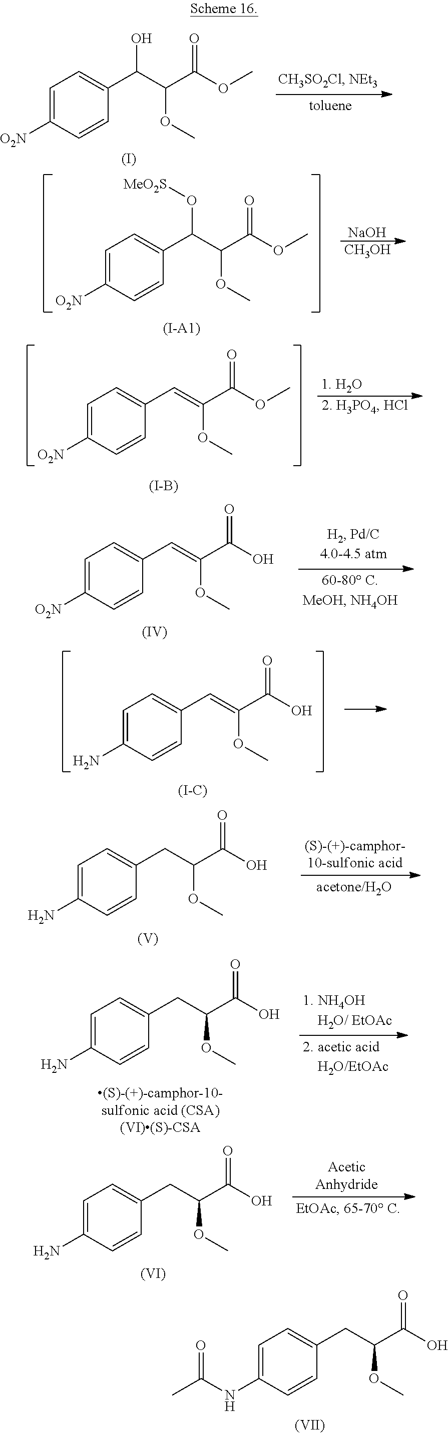

### Preparation of 2-methoxy-3-(4-nitrophenyl)acrylic acid (IV):

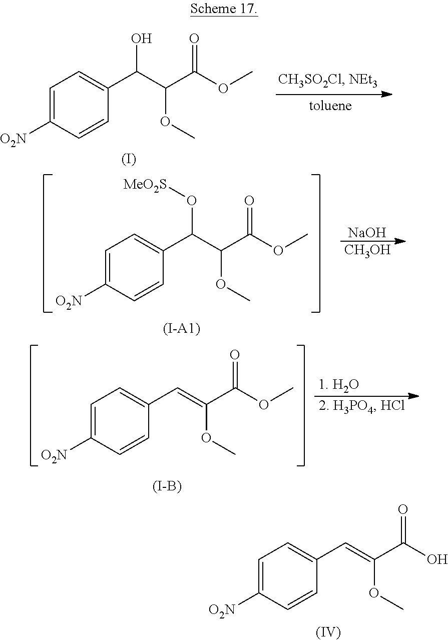

TABLE 15
Batch formula at production scale (synthesis of compound (IV))

| Material | Factor* | Amount (kg) |
|---|---|---|
| methyl 3-hydroxy-2-methoxy-3- | 1.0 | All solution obtained |
| (4-nitrophenyl)propanoate, | | in Scheme 15 |
| solution in toluene | | |
| triethylamine | 0.9 | 342 |
| methanesulfonyl chloride | 0.8 | 304 |
| deionized water | 3.0 | 1140 |
| Toluene | 0.15 | 57 |
| methanol | 3.1 | 1178 |
| sodium hydroxide 30% | 1.86 | 707 |
| deionized water | 3.88 | 1474 |
| hydrochloric acid 37% | 0.74 | 281 |
| phosphoric acid 85% | 0.16 | 61 |
| phosphoric acid 85% | — | to pH ≤3.0 |
| deionized water | 4.0 | 1520 |
| toluene | 2.4 | 912 |

*factor is referenced to the amount of 4-nitrobenzaldehyde used

The methyl 3-hydroxy-2-methoxy-3-(4-nitrophenyl)propanoate (I) in toluene solution in a stainless steel reactor was cooled to 0 to 10° C. while flushing with nitrogen, and while maintaining the temperature at 0 to 10° C. triethylamine (342 kg) and methanesulfonyl chloride (304 kg) were added, and the reaction stirred at 0 to 10° C. for 60 minutes. Deionized water (1140 kg) was slowly added, and the resulting mass was stirred at 55 to 60° C. for 10 minutes, after which time stirring was stopped and the phases were allowed to separate. The aqueous phase was eliminated and the organic phase was filtered, washing the filter with toluene (57 kg). The filtered solution was heated to 55 to 60° C. for 10 minutes. Stirring was stopped and the phases were allowed to separate; the possible aqueous phase was sent to the waste. Under vacuum and under stirring distil an aliquot of the toluene taking into consideration the following proportion: for 380 kg of nitrobenzaldehyde used in Scheme 15, approximately 38 kg of toluene was distilled and this aliquot of distilled toluene was sent to waste. The toluene was removed by distillation under vacuum and without exceeding 60° C., to obtain an oily residue. The resulting residue was then treated with methanol (1178 kg), flushed with nitrogen, cooled to 20 to 30° C. and treated slowly with 30% sodium hydroxide (707 kg). The reaction was stirred at 20 to 30° C. for 3 hours. Deionized water (1474 kg) was added, and the solution heated to 60 to 65° C. At this temperature, 37% hydrochloric acid (281 kg) and 85% phosphoric acid (61 kg) were slowly added to precipitate the product. If necessary, 85% phosphoric acid was added to obtain pH≤3.0. The resulting precipitated product was stirred at 60 to 65° C. for at least 30 minutes then cooled to 35 to 40° C., and stirred for at least 30 minutes. The mixture was centrifuged washing with deionized water (1520 kg) and then washed with toluene (912 kg). The wet product (about 786 kg) was used in the next step.

### Preparation of 3-(4-aminophenyl)-2-methoxypropanoic acid (V)

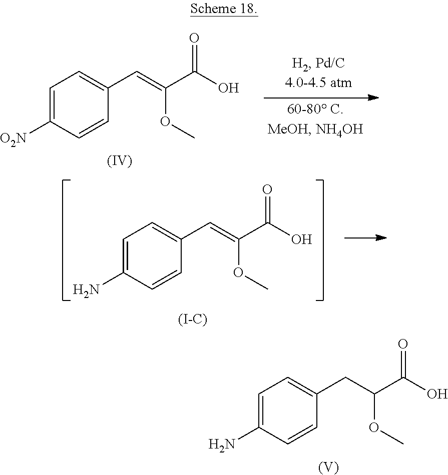

TABLE 16
Batch formula at production scale (synthesis of compound (V))

| Material | Factor* | Amount (kg) |
|---|---|---|
| 2-methoxy-3-(4-nitrophenyl)acrylic | 1.0 | 786 |
| acid, wet | | |
| methanol | 3.0 | 1140 |
| Ammonia 30% | 0.3 | 114 |
| deionized water | 0.2 | 76 |
| palladium on carbon 5% (containing | 0.1 | 38 |
| 50% water) or palladium on carbon | | |
| 5% (recycled) | | |
| deionized water | 0.05 | 19 |
| deionized water | 0.05 | 19 |
| deionized water | 0.4 | 152 |
| deionized water | 2.0 | 760 |
| hydrochloric acid 37% | 0.32 | 122 |
| acetic acid 80% | 0.22 | 84 |
| ethyl acetate | 1.5 | 570 |
| deionized water | 1.0 | 380 |
| ethyl acetate | 1.0 | 380 |

*factor is referenced to the amount of 4-nitrobenzaldehyde used

### Preparation of Aqueous Suspension of Palladium at 5% on Carbon in Water

A suitable stainless steel reactor was loaded with deionized water (76 kg), 5% palladium on carbon (38 kg) and then the bag was washed with deionized water (19 kg).

### Preparation of 3-(4-aminophenyl)-2-methoxypropanoic acid (V)

A stainless steel reactor flushed with nitrogen (2×) was loaded with wet 2-methoxy-3-(4-nitrophenyl)acrylic acid (IV) (786 kg), methanol (1140 kg) and 30% ammonia (114 kg). After restoring the atmospheric pressure, the mixture was stirred at 20 to 30° C. up to complete dissolution. The resulting solution was flushed with nitrogen (2×) and treated with a previously prepared solution of 5% palladium on carbon in deionized water, and the empty vessel was washed with deionized water (19 kg). The reaction was flushed with nitrogen (2×), heated to 60 to 80° C. then charged with hydrogen until a pressure of 4.0 to 4.5 atm was obtained. The reaction was allowed to proceed, maintaining the pressure between 4.0 and 4.5 atm until hydrogen consumption ceased and the reaction was confirmed complete. The reactor was restored to atmospheric pressure and the reaction was cooled to 20 to 30° C., filtered through sparkler filter, flushed with nitrogen (2×) and treated with deionized water (152 kg) that was combined with the solution containing the product. Without exceeding 50° C., the solvent was distilled under stirring and under vacuum up to oily residue. The residue was treated with deionized water (760 kg), heated to 65 to 70° C. and the product precipitated by adding 37% hydrochloric acid (122 kg) and 80% acetic acid (84 kg). The mixture was stirred at 65 to 70° C. for at least 10 minutes; at this stage it was confirmed that the pH was between 3.8 and 4.2 (not more than 4.2), and then the mixture was treated with ethyl acetate (570 kg), flushed with nitrogen and stirred at 65 to 70° C. for at least 15 minutes. The reaction mass was cooled to 15 to 20° C. and stirred for at least 30 minutes. The suspension was centrifuged portion-wise washing with deionized water (380 kg) and ethyl acetate (380 kg). The moist product was granulated, and dried at 60 to 70° C. to obtain 3-(4-aminophenyl)-2-methoxypropanoic acid (V) (˜295 kg).

### Preparation of (S)-(−)-3-(4-Aminophenyl)-2-methoxypropionic acid S-(+)-camphor-10-sulfonic acid salt

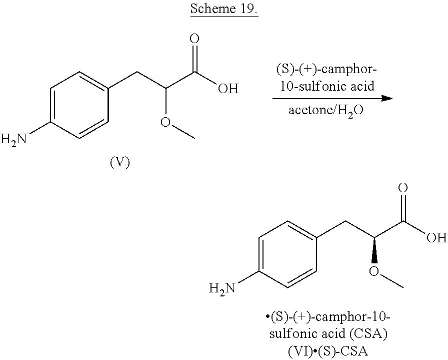

Starting material 3-(4-aminophenyl)-2-methoxypropanoic acid deriving from two batches were combined (one entire batch and an aliquot from a second one) to obtain the total amount of 400 kg of 3-(4-aminophenyl)-2-methoxypropanoic acid. This quantity is equivalent to the amount that would have been obtained starting from 500 kg of 4-nitrobenzaldehyde.

TABLE 17
Batch formula at production scale (synthesis
of compound (VI)•(S)-CSA)

| Material | Factor* | Amount (kg) |
|---|---|---|
| 3-(4-aminophenyl)-2- | 1.0 | 400 |
| methoxypropanoic acid | | |
| (S)-(+)-10-camphor-sulfonic | 1.2321 | 493 |
| acetone | 2.0 | 800 |
| deionized water | 0.16 | 64 |
| acetone | 2.0 | 800 |
| acetone | 1.5 | 600 |

*factor is referenced to the amount of 3-(4-aminophenyl)-2-methoxypropanoic acid (dried) used

A stainless steel reactor was loaded with 3-(4-aminophenyl)-2-methoxypropanoic acid (V) (400 kg) and (S)-(+)-camphor-10-sulfonic acid (493 kg); nitrogen was flushed and acetone (800 kg) was added. Deionized water (64 kg) was added. The reaction was heated to approximately 58° C. for 1 hour (reflux, dissolution does not occur). Acetone (800 kg) was added (temperature approximately 58° C.) and the reaction mass was kept at reflux (approx. 58° C.) for 1 hour; the reaction mass was then cooled to 37 to 42° C. and was centrifuged portion-wise washing with acetone (600 kg) (between every centrifugation step, the suspension aliquot that was not involved in the centrifugation was first heated tan kept at 45 to 50° C. and then, before the subsequent centrifugation, the suspension was cooled again to 37 to 42° C.). The centrifuged product (S)-(−)-3-(4-aminophenyl)-2-methoxypropionic acid S-(+)-camphor-10-sulfonic acid salt (VI)⋅(S)-CSA (wet weight: 470 kg, 409.7 as dried) was used directly in the next step.

### Preparation of (S)-(−)-3-(4-Aminophenyl)-2-methoxypropionic acid (VI)

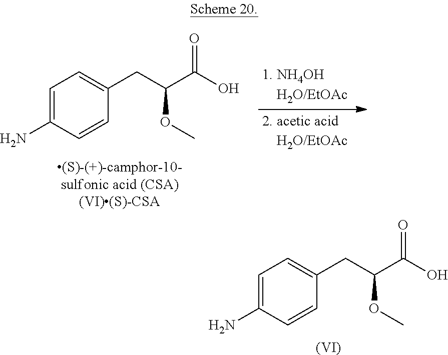

TABLE 18
Batch formula at production scale (synthesis of compound (VI))

| Material | Factor* | Amount (kg) |
|---|---|---|
| deionized water | 2.0 | 819 |
| (S)-(−)-3-(4-Aminophenyl)-2- | 1.0 | 470 (409.7 |
| methoxypropionic acid S-(+)- | | kg as dried) |
| camphor-10-sulfonic acid salt, | | |
| wet | | |
| deionized water | 0.2 | 82 |
| ethyl acetate | 1.0 | 410 |
| ammonia 30% | 0.1257 | 51 |
| acetic acid 80% | 0.05 | 20.5 |
| deionized water | 0.7 | 287 |
| ethyl acetate | 0.7 | 287 |

*factor is referenced to the amount of (S)-(−)-3-(4-aminophenyl)-2-methoxypropionic acid S-(+)-camphor-10-sulfonic acid salt used (as dried).

A stainless steel reactor was charged with deionized water (819 kg) and wet ((S)-(−)-3-(4-aminophenyl)-2-methoxypropionic acid S-(+)-camphor-10-sulfonic acid salt (VI)⋅(S)-CSA (wet weight: 470 kg, 409.7 as dried), flushed with nitrogen and heated for at least 10 minutes at 20 to 40° C. until complete dissolution occurred. The solution was filtered washing with deionized water (82 kg), then ethyl acetate (410 kg). The resulting solution was flushed with nitrogen, heated to 55 to 60° C. and 30% ammonia (51 kg) was added to precipitate the product. The resulting mixture was stirred for at least 15 minutes at 55 to 60° C. then 80% acetic acid (20.5 kg) was added through a filter and the reaction flushed with nitrogen and stirred at 55 to 60° C. for at least 30 minutes then cooled down to 2-7° C. and stirred for at least 1 hour. The reaction mass was centrifuged portion-wise and washed with deionized water (287 kg), ethyl acetate (287 kg) to deliver wet (S)-(−)-3-(4-aminophenyl)-2-methoxypropionic acid (VI) (wet weight 162 kg, 138.8 kg as dried), which was used (wet) directly in the next step.

### Preparation of (S)-(−)-3-(4-Acetamidophenyl)-2-methoxypropionic acid (VII)

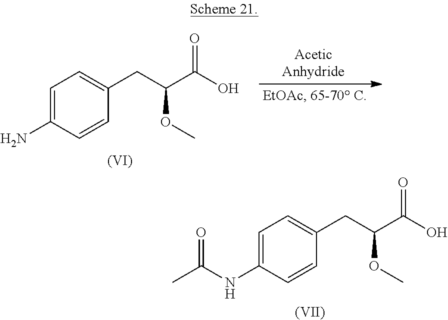

TABLE 19
Batch formula at production scale (synthesis of compound (VII))

| Material | Factor* | Amount (kg) |
|---|---|---|
| Wet (S)-(−)-3-(4-Aminophenyl)- | 1.0 | 162 (138.8 |
| 2- methoxypropionic acid | | kg as dried) |
| ethyl acetate | 1.5 | 208 |
| acetic anhydride | 0.65 | 90 |
| ethyl acetate | 0.5 | 4.80 |
| deionized water | 1.5 | 208 |

*factor is referenced to the amount of (S)-(−)-3-(4-aminophenyl)-2-methoxypropionic acid used (as dried)

A stainless steel reactor was charged with wet (S)-3-(4-aminophenyl)-2-methoxypropanoic acid (VI) (wet weight 162 kg, 138.8 kg as dried) and ethyl acetate (208 kg), under nitrogen. The mixture was heated to 65 to 70° C. and acetic anhydride (90 kg) was added through a cartridge filter over about 15 minutes. The reaction was stirred at 65 to 70° C. for 90 minutes and, once the reaction end was detected, continuously stirred until formation of a precipitate. The mixture was cooled to 10 to 20° C. and stirred at 10 to 20° C. for 30 minutes, then centrifuged and the resulting solid washed with ethyl acetate (69 kg), and deionized water (208 kg). The resulting product was dried at 60 to 70° C. for 19 hours, and milled to obtain (S)-3-(4-acetamidophenyl)-2-methoxypropanoic acid (VII) (154.5 kg).

(S)-(−)-3-(4-Acetamidophenyl)-2-methoxypropionic acid (VII): IR: 3319, 2888, 2825, 1718, 1633, 1599, 1548, 1107 cm⁻¹; LCMS (+)ESI: m/z calculated for C₁₂H₁₅NO₄: 237, found 236 (M-H)⁻; ¹H NMR (600 MHz, DMSO-d₆): δ 12.70 (s, 1 H), 9.85 (s, 1 H), 7.45 (d, 2 H), 7.12 (d, 2 H), 3.90 (dd, 1 H), 3.20 (s, 3 H), 2.80 (ABd, 2 H), 2.00 (s, 3 H). ¹³C NMR (600 MHz, DMSO-d₆): δ 24.09 (CH₃—CO—NH—), 37.94 (—C—CH₂—C—), 57.47 (CH₃—O—), 80.98 (—C—CH(O)—C—), 119.25 (2C, aromatic), 129.65 (2C, aromatic), 132.24 (1C, aromatic), 137.87 (1C, aromatic), 168.47 (CH₃—CO—NH—), 173.21 (—COOH). EA: C 60.82% (theor. 60.75%), H 6.44% (theor. 6.37%), N 5.91% (theor. 5.90%), O 26.97% (theor. 26.97%). DSC: melting 154-167° C. (onset 159° C.). Specific optical rotation: −22.7. (S)-3-(4-aminophenyl)-2-methoxypropanoic acid (HPLC): 0.06% (HPLC). Chiral purity (HPLC): 99.7% (HPLC). Residual solvents (ethyl acetate): 277 ppm (GC).

### Example 7: X-Ray Crystal Structure Determination of Compound of Formula (VII)

The crystal used in the structural determination was obtained by vapour diffusion of a solution of compound of Formula (VII) and L-proline (2:1) in ethanol, using heptane as antisolvent. Single crystal X-ray diffraction analysis was performed. The results of this analysis are shown below. Refinement on the P2₁2₁2₁ space group lead to an R index of 0.062. The asymmetric unit is composed by two compound of Formula (VII) and two L-proline molecules.

Analysis of the single crystal diffraction data shows that the absolute configuration of the carbon alpha to the carboxylic acid group is (S). Based on these results, the absolute stereochemistry of the compound of Formula (VII) is shown in the structure below.

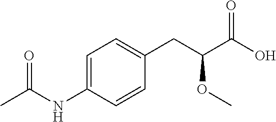 [SMILES: CO[C@@H](Cc1ccc(NC(C)=O)cc1)C(=O)O]

The optical rotation of the same compound, i.e., the compound of Formula (VII), is negative, which means is turns plane-polarized light to the left. The specification of the specific rotation for the pure compound is −26.0° to −19.0°. Accordingly, because the procedure for resolving the racemic mixture always employs the same reagents, characterization that the product obtained has the (S) configuration is confirmed by specific optical rotation.

In addition, the chiral purity (HPLC) of the compound of Formula (VII) can be determined, for example using the method described herein.

The crystal data and structure refinement for the single crystal X-ray diffraction of the compound of Formula (VII):L-proline derivative is set out below.

| Empirical formula | C₁₇H₂₄N₂O₆ | |
|---|---|---|
| Formula weight | 352.38 | |
| Temperature | 296(2) K | |
| Diffractometer | Bruker Smart-Apex (area detector) | |
| Wavelength | 0.71073 Å | |
| Crystal system | Orthorhombic | |
| Space group | P2₁2₁2₁ | |
| Unit cell dimensions | a = 7.7813(5) Å | α = 90° |
| | b = 9.5414(6) Å | β = 90° |
| | c = 49.006(3) Å | γ = 90° |

| | |
|---|---|
| Volume | 3638.4(4) Å³ |
| Z | 8 |
| Density (calculated) | 1.287 Mg/m³ |
| Absorption coefficient | 0.098 mm⁻¹ |
| F(000) | 1504 |
| Crystal size | 0.49 × 0.25 × 0.08 mm³ |
| Theta range for data collection | 0.83 to 28.75°. |
| Index ranges | −10 <= h <= 8, −12 <= |
| | k <= 12, −54 <= l <= 63 |
| Reflections collected | 19513 |
| Independent reflections | 8598 [R(int) = 0.0437] |
| Completeness to theta = 25.00° | 99.7% |
| Absorption correction | Semi-empirical from equivalents |
| Max. and min transmission | 0.992 and 0.777 |
| Refinement method | Full-matrix least-squares on F² |
| Data/restraints/parameters | 8598/0/474 |
| Goodness-of-fit on F² | 0.984 |
| Final R indices [I > 2sigma(I)] | R1 = 0.0623, wR2 = 0.1517 |
| R indices (all data) | R1 = 0.1217, wR2 = 0.1843 |
| Absolute structure parameter | −0.8(13) |
| Largest diff. peak and hole | 0.382 and −0307 e · Å⁻³ |
| Computing programs (Bruker) | Smart 5.6/Saint 5.0/Shelxtl-NT 6.1 |

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### Equivalents

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A process for preparing a substantially optically pure compound of Formula (VII): 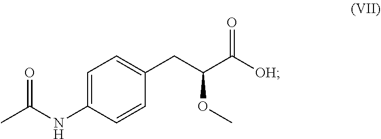 [SMILES: CO[C@@H](Cc1ccc(NC(C)=O)cc1)C(=O)O]
the process comprising:
reacting a compound of Formula (I): 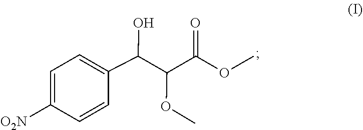 [SMILES: COC(=O)C(OC)C(O)c1ccc([N+](=O)[O-])cc1]
with an activating agent, in the presence of an amine base, to form an intermediate of Formula (I-A): 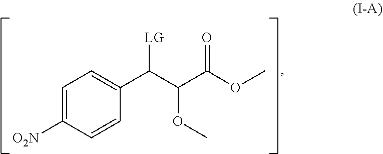
wherein LG is a leaving group;
treating the intermediate of Formula (I-A) with a base solution in the presence of an alcohol solvent, to eliminate the leaving group and thereby forming an intermediate of Formula (I-B): 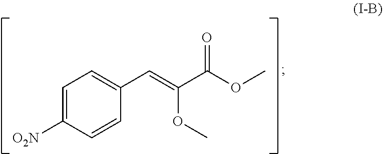
hydrolyzing the intermediate of Formula (I-B) to form a compound of Formula (IV): 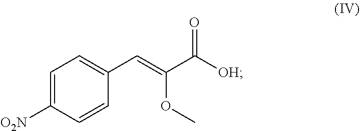 [SMILES: CO/C(=C\c1ccc([N+](=O)[O-])cc1)C(=O)O]
hydrogenating the compound of Formula (IV) to form a compound of Formula (V): 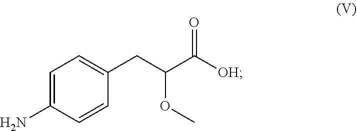
resolving the compound of Formula (V) to form a substantially optically pure compound of Formula (VI): 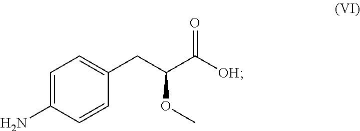
and
acylating the compound of Formula (VI) to form the compound of Formula (VII), wherein the substantially optically pure compound of Formula (VII) is at least 95% of the enantiomer: 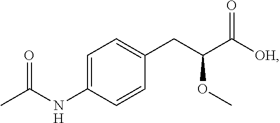
expressed as a percentage of both enantiomers.

2. The process of claim 1, wherein reacting a compound of Formula (I) with an activating agent comprises reacting in the presence of a solvent selected from the group consisting of toluene, dichloromethane, tetrahydrofuran, diethyl ether, 2-methyltetrahydrofuran, and acetonitrile.

3. (canceled)

4. The process of claim 2, wherein the solvent is toluene.

5. (canceled)

6. The process of claim 1, wherein the amine base is selected from the group consisting of triethylamine, N,N-diisopropylethylamine, and pyridine.

7. The process of claim 6, wherein the amine base is triethylamine.

8. The process of claim 1, wherein the activating agent is a sulfonylating agent, or a halogenating agent.

9. The process of claim 1, wherein the activating agent is selected from the group consisting of a methanesulfonyl chloride, p-toluenesulfonyl chloride, p-bromobenzenesulfonyl chloride, phenyl triflimide, triflic anhydride, and nonafluorobutanesulfonic anhydride.

10. The process of claim 1 any one of claims 1 9, wherein the activating agent is methanesulfonyl chloride.

11. The process of claim 1, wherein the leaving group is selected from the group consisting of —OSO₂-aryl, —OSO₂—C₁₋₄alkyl, chloro, bromo, and iodo; wherein C₁₋₄alkyl and aryl may be optionally substituted with one or more substituents each independently selected, for each occurrence, from the group consisting of fluoro, bromo, and —CH₃.

12. The process of claim 11, wherein the leaving group is —OSO₂-phenyl or —OSO₂—C₁₋₄alkyl.

13. (canceled)

14. The process of claim 1, wherein the leaving group is —OSO₂Me.

15. (canceled)

16. The process of claim 1, wherein the alcohol solvent comprises methanol.

17. (canceled)

18. The process of claim 1, wherein the base solution comprises sodium hydroxide.

19. (canceled)

20. The process of claim 1, wherein hydrolyzing the intermediate of Formula (I-B) to form a compound of Formula (IV) comprises:
(i) contacting the intermediate of Formula (I-B) with an alkali hydroxide and water; and
neutralizing to form the compound of Formula (IV).

21-23. (canceled)

24. The process of claim 1, wherein hydrogenating the compound of Formula (IV) to form a compound of Formula (V) comprises contacting the compound of Formula (IV) with hydrogen and a catalyst.

25-26. (canceled)

27. The process of claim 1, wherein resolving the compound of Formula (V) to form a substantially optically pure compound of Formula (VI) comprises:
(a) resolving a compound of Formula (V) in the presence of a chiral acid thereby forming a chiral salt of the compound of Formula (VI); and
(b) neutralizing the chiral salt of the compound of Formula (VI) thereby forming the compound of Formula (VI).

28-29. (canceled)

30. The process of claim 27, wherein the chiral salt of the compound of Formula (VI) is:

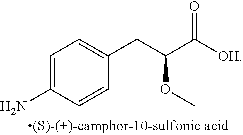

31. (canceled)

32. The process of claim 27, wherein neutralizing comprises contacting the chiral salt of the compound of Formula (VI), with (i) an aqueous base; and then (ii) acidifying the solution by adding an acid.

33. (canceled)

34. The process of claim 1, wherein acylating comprises contacting the compound of Formula (VI) with an acylating agent in the presence of an organic solvent selected from the group consisting of ethyl acetate, tetrahydrofuran, diethyl ether, dichloromethane, and toluene.

35-39. (canceled)

40. The process of claim 1, wherein the substantially optically pure compound of Formula (VII) is at least 98% of the enantiomer: 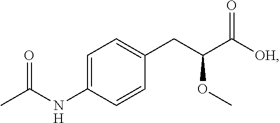
expressed as a percentage of both enantiomers.

41-51. (canceled)
